SEQUENCE LISTING (2 sequences)

SEQ ID NO: 1 - RNA, 21 nt, synthetic construct
  misc_feature 1..21: note = Synthetic
  source 1..21: mol_type = other RNA
gctcttctgtctactgaactt

SEQ ID NO: 2 - RNA, 23 nt, synthetic construct
  misc_feature 1..23: note = Synthetic
  source 1..23: mol_type = other RNA
aagttcagtagacagaagagcgt

(19) United States
(12) Patent Application Publication
(10) Pub. No.: US 2026/0283974 A1
(43) Pub. Date: Sep. 24, 2026

(54) METHODS OF LYOPHILIZING LIPID NANOPARTICLES

(71) Applicant: TAKEDA PHARMACEUTICAL COMPANY LIMITED, Osaka (JP)

(72) Inventors: Kanika Suri, Lexington, MA (US); Amit Singh, Lexington, MA (US); Amiji M. Mansoor, Lexington, MA (US)

(21) Appl. No.: 19/472,550

(22) PCT Filed: Apr. 5, 2024
(86) PCT No.: PCT/US2024/023468

§ 371 Date: Oct. 3, 2025

Related U.S. Application Data
(60) Provisional application No. 63/457,770, filed on Apr. 6, 2023.

(51) Int. Cl. A61K 9/51; A61K 9/00; A61K 9/19; A61K 31/7105; A61P 1/04

(52) U.S. Cl. CPC A61K 9/5138; A61K 9/0053; A61K 9/19; A61K 9/5123; A61K 9/5192; A61K 31/7105; A61P 1/04

(57) ABSTRACT

This disclosure features novel lipid nanoparticle formulations and uses thereof. The lipid nanoparticle (“LNP”) includes an encapsulated therapeutic agent and an aqueous solution comprising a salt and an anionic polymer, wherein the salt and the anionic polymer are dissolved in the aqueous solution, thereby forming polymer coated lipid nanoparticle (“PCLNP”). Lipid nanoparticles of this disclosure are useful in the process of lyophilization or freeze drying and decrease nanoparticle aggregation and maintain efficacy once reconstituted.

### RELATED APPLICATIONS

The instant application claims priority to U.S. Provisional Application No. 63/457,770, filed Apr. 6, 2023, the entire contents of which are expressly incorporated by reference herein.

### BACKGROUND

Intestinal diseases such as Crohn's disease, irritable bowel syndrome, ulcerative colitis and gastrointestinal cancer affect millions of people worldwide. Current therapies are mainly focused on treatment of symptoms and are often expensive and ineffective in the long term.

Potential advancements in treatment of intestinal diseases have included the use of lipidoid nanoparticles (LNPs) as vehicles for delivering short interfering RNA (siRNA) which can reduce or eliminate production of harmful proteins. The preferred route of administration of such compositions is oral administration, but nucleic acids do not survive the traverse of the gastrointestinal tract. Exposure to digestive enzymes, bile acids, mucus and extremes of acidity and alkalinity eliminates the ability to effectively reach the intestinal cells and prevent the production of the inflammatory proteins. See R. Ball et al., “Oral delivery of siRNA lipid nanoparticles: Fate in the GI tract,” Scientific Reports 8:2178 (2018).

Lyophilization or freeze drying of the LNPs has been investigated as a way of possibly protecting the siRNA during traverse of the GI tract to permit interaction of the siRNA with the epithelial cells of the intestine and effective silencing of the genes which produce the harmful proteins. For example, see R. Ball et al., “Achieving long-term stability of lipid nanoparticles: examining the effect of pH, temperature, and lyophilization,” Int. J. Nanomedicine, 12:305 (2017). There the researchers found that the use of lyoprotectants such as sucrose or trehalose during the lyophilization process was necessary to improve stability, gene silencing ability, siRNA entrapment, particle size and monodispersity of the LNPs. The conclusion was that if freezing of the LNPs is desired, the addition of 20% (w/v) of sucrose or trehalose was found to be necessary to prevent nanoparticle aggregation and loss of efficacy. The alternative to use of sugars was to use 30% of ethanol in the liquid used to reconstitute the lyophilized particles. That approach was impractical, since the reconstituted particles would have to be dialyzed before being given to patients.

### SUMMARY OF THE INVENTION

Accordingly, in one aspect, the present invention provides a composition comprising at least one lipid nanoparticle (“LNP”) encapsulating a therapeutic agent and an aqueous solution comprising a salt and an anionic polymer, wherein the salt and the anionic polymer are dissolved in the aqueous solution.

In one embodiment, the anionic polymer is bound to the surface of the lipid nanoparticle, thereby forming a polymer coated lipid nanoparticle (“PCLNP”). Preferably the polymer binding to the LNP occurs due to the negative nature of the anionic polymer and the positive nature of the lipid nanoparticles (LNPs).

In one embodiment, the polymer coat covers at least 10%, at least 20%, at least 30%, at least 40%, at least 50%, at least 60%, at least 70%, at least 80%, at least 90%, at least 95%, at least 98%, or at least 100% of the surface of the lipid nanoparticle. In some embodiments, the polymer is in excess of the surface of the lipid nanoparticle.

In one embodiment, the anionic polymer is a methacrylic acid polymer. Eudragit® is the trademark for a diverse range of polymethacrylate-based copolymers. It includes anionic, cationic, and neutral copolymers based on copolymers derived from esters of acrylic and methacrylic acid. Preferably the amount of ionic polymer based on the amount of LNPs on a w/w basis is from about 1 to about 100, more preferably from about 1 to about 12, more preferably from about 1.18 to about 11.75. On a volume basis, the amount of ionic polymer is about 50 μL to about 500 μL per mL of LNP, more preferably about 50 μL to about 150 μL per mL of LNP.

In one embodiment, the PCLNP has a negative zeta potential. In one embodiment, the zeta potential is between about −0.1 mV to about −50 mV, about −1.0 mV to about −45 mV, about −2.0 mV to about −30 mV, about −10 mV to about −20 mV, or about −5.0 mV to about −10 mV. In one embodiment, the zeta potential is at least −0.1 mV, at least −0.5 mV, at least −1.0 mV, at least −2.0 mV, at least −5.0 mV, at least −10 mV, at least −15 mV, at least −20 mV, at least −25 mV, at least −30 mV, at least −35 mV, at least −40 mV, at least −45 mV, or at least −50 mV. In one embodiment, the zeta potential is at most −0.1 mV, at most −0.5 mV, at most −1.0 mV, at most −2.0 mV, at most −5.0 mV, at most −10 mV, at most −15 mV, at most −20 mV, at most −25 mV, at most −30 mV, at most −35 mV, at most −40 mV, at most −45 mV, or at most −50 mV.

The polymer coated lipid nanoparticles in the aqueous compositions in accordance with the present invention preferably have a negative zeta potential. Preferably the zeta potential is between about −0.1 mV to about −50 mV. Preferably the range is from −10 mV to −50 mV. In the unlyophilized compositions, the zeta potential is determined by measuring the potential between the surface of the solid being tested and its liquid medium. Lyophilized PCLNPs need to be reconstituted before their zeta potential can be determined. Preferably zeta potential is determined both before and after lyophilization.

The difference in zeta potential is assessed with the following formula:

100×(zeta potential before lyophilization/zeta potential after lyophilization and reconstitution)=% difference

In one embodiment, the polydispersity index (PDI) of the PCLNPs in the composition is less than 1.0, less than 0.9, less than 0.8, less than 0.7, less than 0.6, less than 0.5, less than 0.4, less than 0.3, less than 0.2, or less than 0.1. In one embodiment, the PDI is less than 0.2.

In one embodiment, the difference of the size of the PCLNP before and after the lyophilization is less than 40%, less than 35%, less than 30%, less than 25%, less than 20%, less than 15%, less than 10%, or less than 5%.

Particle size is assessed before and after the lyophilization of the PCLNP. For assessment after lyophilization, the particles have to be reconstituted. The difference in size is assessed with the following formula:

$$100 \times ( SIZE ( nm ) before lyophilization / SIZE after lyophilization and reconstitution ) = \% difference$$

In one embodiment, the difference of the PDI of the PCLNP before and after the lyophilization is less than 40%, less than 35%, less than 30%, less than 25%, less than 20%, less than 15%, less than 10%, or less than 5%.

The lyophilized material has to be reconstituted before measurement. PDI is defined as the standard deviation of the particle diameter distribution divided by the mean particle diameter. PDI is used to estimate the average uniformity of a particle solution. Larger PDI values correspond to a larger size distribution in the particle sample.

$$100 \times ( PDI before lyophilization / PDI after lyophilization and reconstitution ) = \% difference$$

In one embodiment, the nucleic acid molecule is DNA or RNA. In one embodiment, the nucleic acid molecule is RNA. In one embodiment, the RNA is mRNA, microRNA (miRNA), or short interfering RNA (siRNA). Preferably the RNA segments encapsulated by the LNPs cause a pharmaceutical effect on patients, and the composition of matter can be utilized as a pharmaceutical composition, suitable for administration to a patient to alleviate some disease or disease symptom.

In one embodiment, the nucleic acid molecule is between about 10 nucleotides (nt) and 20 kilobases (kb), about 500 nt to about 20 kb, about 1 kb to about 20 kb, about 2 kb to about 20 kb, about 3 kb to about 20 kb, about 4 kb to about 20 kb, about 5 kb to about 20 kb, about 6 kb to about 20 kb, about 7 kb to about 20 kb, about 8 kb to about 20 kb, about 9 kb to about 20 kb, about 10 kb to about 20 kb, about 15 kb to about 20 kb, about 10 nt and 15 kb, about 500 nt to about 15 kb, about 1 kb to about 15 kb, about 2 kb to about 15 kb, about 3 kb to about 15 kb, about 4 kb to about 15 kb, about 5 kb to about 15 kb, about 6 kb to about 15 kb, about 7 kb to about 15 kb, about 8 kb to about 15 kb, about 9 kb to about 15 kb, about 10 kb to about 15 kb, about 10 nt and 10 kb, about 500 nt to about 10 kb, about 1 kb to about 10 kb, about 2 kb to about 10 kb, about 3 kb to about 10 kb, about 4 kb to about 10 kb, about 5 kb to about 10 kb, about 6 kb to about 10 kb, about 7 kb to about 10 kb, about 8 kb to about 10 kb, about 9 kb to about 10 kb, about 10 nt and 8 kb, about 500 nt to about 8 kb, about 1 kb to about 8 kb, about 2 kb to about 8 kb, about 3 kb to about 8 kb, about 4 kb to about 8 kb, about 5 kb to about 8 kb, about 6 kb to about 8 kb, about 7 kb to about 8 kb, about 10 nt and 6 kb, about 500 nt to about 6 kb, about 1 kb to about 6 kb, about 2 kb to about 6 kb, about 3 kb to about 6 kb, about 4 kb to about 6 kb, about 5 kb to about 6 kb, about 10 nt and 5 kb, about 500 nt to about 5 kb, about 1 kb to about 5 kb, about 2 kb to about 5 kb, about 3 kb to about 5 kb, about 4 kb to about 5 kb, about 10 nt and 4 kb, about 500 nt to about 4 kb, about 1 kb to about 4 kb, about 2 kb to about 4 kb, about 3 kb to about 4 kb, about 10 nt and 3 kb, about 500 nt to about 3 kb, about 1 kb to about 3 kb, about 2 kb to about 3 kb, about 10 nt and 2 kb, about 500 nt to about 2 kb, about 1 kb to about 2 kb, about 10 nt and 1 kb, about 500 nt to about 1 kb, or about 10 nt to about 500 nt. Preferably, the nucleic acid is RNA, e.g., siRNA. Preferably, the siRNA is about 20 nt to about 25 nt.

In some embodiments, the phrase “N/P ratio” refers to the ratio of positively-chargeable lipid, lipidoid or polymer amine (N=nitrogen) groups to negatively-charged nucleic acid phosphate (P) groups. In some embodiments, the nucleic acid is RNA. In some embodiments, the nucleic acid is DNA. In some embodiments, the N/P ratio is about 1 to about 35, about 1 to about 30, about 1 to about 25, about 1 to about 20, about 1 to about 15, about 1 to about 10, about 1 to about 5, about 5 to about 35, about 5 to about 30, about 5 to about 25, about 5 to about 20, about 5 to about 15, about 5 to about 10, about 10 to about 35, about 10 to about 30, about 10 to about 25, about 10 to about 20, about 10 to about 15, about 15 to about 35, about 15 to about 30, about 15 to about 25, about 15 to about 20, about 20 to about 35, about 20 to about 30, about 20 to about 25, about 25 to about 35, about 25 to about 30, or about 30 to about 35. Preferably, the nucleic acid is RNA and the N/P ratio is about 5 to about 30. In some embodiments, the nucleic acid is DNA and the N/P ratio is about 15 to about 20.

In one embodiment, the lipid nanoparticle comprises at least one lipid selected from the group consisting of a cationic lipid, an anionic lipid, a neutral lipid, a zwitterionic lipid, an ionizable lipid, a helper lipid, a cholesterol, a PEG-lipid, phospholipid, and a combination thereof. In one embodiment, the lipid nanoparticle comprises an ionizable lipid and a cationic lipid.

In one embodiment, the combination of the ionizable lipid and the cationic lipid is more than about 20%, more than about 30%, more than about 40%, more than about 50%, or more than about 60% of the total lipid in the lipid nanoparticle.

In one embodiment, the ionizable lipid is a primary amino lipid, a secondary amino lipid a tertiary amino lipid, or a multivalent lipid. In one embodiment, the multivalent lipid is selected from the group consisting of N1-[2-((1S)-1-[(3-aminopropyl)amino]-4-[di(3-amino-propyl)amino]butylcarboxamido)ethyl]-3,4-di[oleyloxy]-benzamide (MVL5), 2,3-dioleyloxy-N-[2-(sperminecarboxamido)ethyl]-N,N-dimethyl-1-propanaminium HCl salt (DOSPA), and N4-Cholesteryl-Spermine HCl Salt (GL67). In one embodiment, the cationic lipid is a quaternary amino lipid. In one embodiment, the cationic lipid is 1,2-di-O-octadecenyl-3-trimethylammonium propane (DOTMA). In one embodiment, a cationic lipid is selected from the group consisting of 3β-[N—(N′,N′-dimethylaminoethane)-carbamoyl]cholesterol hydrochloride (DC-Cholesterol), DOTAP:DOPE (1:1 w/w), DOTAP:DOPE (1:1, w/w)+0.1 wt. % NBD-DOPE, 1-oleoyl-2-[6-[(7-nitro-2-1,3-benzoxadiazol-4-yl)amino]hexanoyl]-3-trimethylammonium propane (chloride salt) (fluorescent DOTAP), N-(2-hydroxyethyl)-N,N-dimethyl-2,3-bis(oleoyloxy) propan-1-aminium bromide (DORI), O,O′-ditetradecanoyl-N-(α-trimethylammonioacetyl) diethanolamine chloride (DC-6-14), 1,2-dilauroyl-sn-glycero-3-ethylphosphocholine (chloride salt) (12:0 EPC), 1,2-dimyristoyl-sn-glycero-3-ethylphosphocholine (chloride salt) (14:0 EPC), 1,2-dipalmitoyl-sn-glycero-3-ethylphosphocholine (chloride salt) (16:0 EPC), 1,2-distearoyl-sn-glycero-3-ethylphosphocholine (chloride salt) (18:0 EPC), 1,2-dioleoyl-sn-glycero-3-ethylphosphocholine (chloride salt) (18:1 EPC), 1-palmitoyl-2-oleoyl-sn-glycero-3-ethylphosphocholine (chloride salt) (16:0-18:0 EPC), 1,2-dimyristoleoyl-sn-glycero-3-ethylphosphocholine (Tf salt) (14:1 EPC), Dimethyldioctadecylammonium (Bromide Salt) (18:0 DDAB), 1,2-dimyristoyl-3-trimethylammonium-propane (chloride salt) (14:0 TAP), 1,2-dipalmitoyl-3-trimethylammonium-propane (chloride salt) (16:0 TAP), 1,2-stearoyl-3-trimethylammonium-propane (chloride salt) (18:0 TAP), 1,2-dioleoyl-3-trimethylammonium-propane (chloride salt) (18:1 TAP (DOTAP)), 1,2-dioleoyl-3-trimethylammonium-propane (methyl sulfate salt) (18:1 TAP (DOTAP, MS salt)), and 1,2-di-O-octadecenyl-3-trimethylammonium propane (chloride salt) (DOTMA).

The LNP compositions are preferably prepared by microfluidic mixing. Preferred other components of the compositions may include C12-200 (a cationic lipid, DOTMA (a polar lipid), cholesterol, DSPC (distearolylphosphatidylcholine, a zwitterionic phospholipid), and/or DMG-PEG (2k)(1.2-dimyristoyl-rac-glycero-3-methoxypolyethlene glycol-2000, a synthetic polar lipid). In a preferred combination, with the amount of lipid at 2 mg/ml, all of those ingredients are included, at molecular ratios of about 30:20:38.5:10:1.5 mol %, respectively.

In one embodiment, the lipid nanoparticle net charge is negative at pH 7. In one embodiment, the lipid nanoparticle net charge is positive at pH 7. In one embodiment, the lipid nanoparticle net charge is neutral at pH 7.

In one embodiment, the aqueous solution comprising a salt is selected from the group consisting of HEPES (4-(2-hydroxyethyl)-1-piperazineethanesulfonic acid), PBS (phosphate buffered saline), TRIS (tris(hydroxymethyl)aminomethane), MES (2-(N-morpholino)ethanesulfonic acid), and citrate buffer. In one embodiment, the aqueous solution comprising a salt is HEPES. In one embodiment, the aqueous solution comprising a salt is PBS.

In one embodiment, the aqueous solution comprising a salt has a pH of about pH 7.0 to about pH 8.0. In one embodiment, the aqueous solution comprising a salt has a pH of about 7.6. Preferably, the buffering nature of the salt is such that the anionic polymer is dissolved in the composition. Depending on the particular polymer utilized, dissolution may occur at pHs from about 5 to about 12, preferably from about 6 to about 9, most preferably from about 6.5 to about 8. Salts which provide buffering in such pH ranges are known in the art.

In one embodiment, the composition further comprises a sugar. In one embodiment, the sugar is disaccharide. In one embodiment, the sugar is selected from the group consisting of lactose, sucrose, trehalose, and a combination thereof. In one embodiment, amount of sugar is between about 0% and about 40%, about 1% to about 40%, about 1% to about 35%, about 1% to about 30%, about 1% to about 25%, about 1% to about 20%, about 1% to about 15%, about 1% to about 10%, about 1% to about 5%, about 5% to about 40%, about 5% to about 35%, about 5% to about 30%, about 5% to about 25%, about 5% to about 20%, about 5% to about 15%, about 5% to about 10%, about 10% to about 40%, about 10% to about 35%, about 10% to about 30%, about 10% to about 25%, about 10% to about 20%, about 10% to about 15%, about 15% to about 40%, about 15% to about 35%, about 15% to about 30%, about 15% to about 25%, about 15% to about 20%, about 20% to about 40%, about 20% to about 35%, about 20% to about 30%, about 20% to about 25%, about 25% to about 40%, about 25% to about 35%, about 25% to about 30%, about 30% to about 40%, about 30% to about 35%, or about 35% to about 40% in the pre-lyophilized solution. In one embodiment, the amount of sugar is at least 5%, at least 10%, at least 20%, or at least 30% in the pre-lyophilized solution. In one embodiment, the amount of sugar is about 5% in the pre-lyophilized solution.

In one embodiment, the composition further comprises a sugar. In one embodiment, the sugar is disaccharide. In one embodiment, the sugar is selected from the group consisting of lactose, sucrose, trehalose, and a combination thereof. In one embodiment, amount of sugar is between about 0% and about 40%, about 1% to about 40%, about 1% to about 35%, about 1% to about 30%, about 1% to about 25%, about 1% to about 20%, about 1% to about 15%, about 1% to about 10%, about 1% to about 5%, about 5% to about 40%, about 5% to about 35%, about 5% to about 30%, about 5% to about 25%, about 5% to about 20%, about 5% to about 15%, about 5% to about 10%, about 10% to about 40%, about 10% to about 35%, about 10% to about 30%, about 10% to about 25%, about 10% to about 20%, about 10% to about 15%, about 15% to about 40%, about 15% to about 35%, about 15% to about 30%, about 15% to about 25%, about 15% to about 20%, about 20% to about 40%, about 20% to about 35%, about 20% to about 30%, about 20% to about 25%, about 25% to about 40%, about 25% to about 35%, about 25% to about 30%, about 30% to about 40%, about 30% to about 35%, or about 35% to about 40% in the lyophilized formulation. In one embodiment, the amount of sugar is at least 5%, at least 10%, at least 20%, or at least 30% in the lyophilized formulation. In one embodiment, the amount of sugar is about 5% in the lyophilized formulation.

In one embodiment, the composition further comprises a sugar. In one embodiment, the sugar is disaccharide. In one embodiment, the sugar is selected from the group consisting of lactose, sucrose, trehalose, and a combination thereof. In one embodiment, amount of sugar is between about 0% and about 40%, about 1% to about 40%, about 1% to about 35%, about 1% to about 30%, about 1% to about 25%, about 1% to about 20%, about 1% to about 15%, about 1% to about 10%, about 1% to about 5%, about 5% to about 40%, about 5% to about 35%, about 5% to about 30%, about 5% to about 25%, about 5% to about 20%, about 5% to about 15%, about 5% to about 10%, about 10% to about 40%, about 10% to about 35%, about 10% to about 30%, about 10% to about 25%, about 10% to about 20%, about 10% to about 15%, about 15% to about 40%, about 15% to about 35%, about 15% to about 30%, about 15% to about 25%, about 15% to about 20%, about 20% to about 40%, about 20% to about 35%, about 20% to about 30%, about 20% to about 25%, about 25% to about 40%, about 25% to about 35%, about 25% to about 30%, about 30% to about 40%, about 30% to about 35%, or about 35% to about 40% in the reconstituted solution. In one embodiment, the amount of sugar is at least 5%, at least 10%, at least 20%, or at least 30% in the reconstituted solution. In one embodiment, the amount of sugar is about 5% in the reconstituted solution.

In one embodiment, the nucleic acid molecule is a short interfering RNA, the anionic polymer is a methacrylic acid polymer, and the lipid nanoparticle comprises at least one lipid selected from the group consisting of a cationic lipid, a polar lipid, a zwitterionic lipid, or a combination thereof.

In one embodiment, the wherein the nucleic acid molecule is a short interfering RNA, the anionic polymer is a methacrylic acid polymer, and the lipid nanoparticle comprises at least one cationic lipid.

In another aspect, the present invention provides a pharmaceutical composition, comprising the composition as disclosed herein, and a pharmaceutically acceptable carrier.

In one embodiment, the composition is lyophilized. Preferably the composition is lyophilized, thus providing lyophilized admixtures of lipid nanoparticles which encapsulate therapeutic agent(s) which comprise nucleotide sequences, such as micro-RNA or short RNA segments, the nanoparticles being coated with an ionic polymer such as a Eudragit®, together with a salt which acts as a buffer in aqueous solution, providing a desired pH range. Preferably the lipid is selected from cationic lipids, polar lipids, zwitterionic lipids or mixtures thereof.

In another aspect, the present invention provides a method of preserving the lyophilized composition as disclosed herein, comprising storing the lyophilized product at a temperature of about 2° C. to about 8° C. In another aspect, the present invention provides a method of preserving the lyophilized composition as disclosed herein comprising storing the lyophilized product at a temperature of about −20° C.

In another aspect, the present invention provides a method of reconstituting the lyophilized composition as disclosed herein comprising adding a liquid medium to the lyophilized composition. In one embodiment, the liquid medium is an aqueous medium.

The present invention also provides a new method of lyophilization, in which, instead of using known lyoprotectants such as sucrose or trehalose, an ionic polymer such as a Eudragit®, preferably Eudragit® S100, is admixed with the other ingredients prior to lyophilization. The addition of the ionic polymer serves to stabilize the lyophilized product against aggregation following reconstitution of the LNPs.

The present invention provides improved methods of lyophilizing lipid nanoparticle compositions, comprising admixing the lipid nanoparticle compositions with ionic polymers prior to freezing the compositions, followed by removal of water in a conventional freeze dryer device. Lyophilized compositions containing a preferred amount ionic polymer can be reconstituted with water, preferably deionized water, without adversely affecting their stability, as indicated by the lack of particle aggregation or loss of the effectiveness of the RNA segments after reconstitution. Alternatively, the lyophilized LNP compositions can be placed in a capsule or otherwise administered to patients needing the treatment provided by the RNA segments.

Preferred lyophilized compositions contain an effective amount of ionic polymer, preferably Eudragit® S100, to stabilize the lyophilized composition against aggregation or degradation on reconstitution. The lyophilized compositions may be used to treat the patients directly, or may be reconstituted prior to treatment, e.g., with deionized water. Prior complicated procedures for reconstitution, e.g. reconstitution with ethanol, followed by dialysis before patient treatment, are unnecessary, because the presence of the ionic polymer stabilizes the LNP composition against aggregation or degradation on reconstitution.

In another aspect, the present invention provides a method of producing a pharmaceutical composition, comprising a) producing lipid nanoparticles from at least one lipid and at least one nucleic acid molecule; and b) mixing an aqueous solution comprising a salt and an anionic polymer with the lipid nanoparticles produced in step a) and a pharmaceutically acceptable carrier.

In one embodiment, the method further comprising c) lyophilizing the resulting mixture produced in the step b).

In one embodiment, the anionic polymer is a methacrylic acid polymer.

In another aspect, the present invention provides a method of stabilizing a lipid nanoparticle for lyophilization, the method comprising: a) producing lipid nanoparticles from the at least one lipid and the at least one nucleic acid molecule; b) mixing an aqueous solution comprising a salt and an anionic polymer with the lipid nanoparticles produced in step a); and c) lyophilizing the resulting mixture, thereby stabilizing the lyophilized lipid nanoparticle.

In one embodiment, the anionic polymer is dissolved in a buffer.

In another aspect, the present invention provides a method of manufacturing a stable pharmaceutical composition, the method comprising: a) dissolving an anionic polymer in an aqueous solution comprising a salt; and b) mixing the dissolved anionic polymer with a lipid nanoparticle comprising at least one nucleic acid.

In one embodiment, the method further comprising c) lyophilizing the resulting mixture produced in the step b).

In one embodiment, step b) further comprises a sugar. In one embodiment, the amount of sugar is about 5%.

In one embodiment, the nucleic acid molecule is DNA or RNA. In one embodiment, the RNA is mRNA, microRNA (miRNA), or short interfering RNA (siRNA).

In one embodiment, the lipid nanoparticle comprises an ionizable lipid and/or a cationic lipid. In one embodiment, the combination of the ionizable lipid and the cationic lipid is more than about 20%, more than about 30%, more than about 40%, more than about 50%, or more than about 60% of the total lipid in the lipid nanoparticle. In one embodiment, the ionizable lipid is a primary amino lipid, a secondary amino lipid, a tertiary amino lipid, or a multivalent lipid. In one embodiment, the cationic lipid is a quaternary amino lipid. In one embodiment, the cationic lipid is 1,2-di-O-octadecenyl-3-trimethylammonium propane (DOTMA).

In one embodiment, the an aqueous solution comprising a salt is a buffer selected from the group consisting of HEPES (4-(2-hydroxyethyl)-1-piperazineethanesulfonic acid), PBS (phosphate buffered saline), TRIS (tris(hydroxymethyl)aminomethane), MES (2-(N-morpholino)ethanesulfonic acid), and citrate buffer. In one embodiment, the aqueous solution comprising a salt has a pH of about pH 7.0 to about pH 8.0.

The present invention provides improved methods of treating patients having a variety of gastro-intestinal diseases, by treating them with the polymer coated lipid nanoparticles encapsulating nucleotides chosen to treat the disease or condition of the patient.

In another aspect, the present invention provides a method of treating a disease or disorder in a subject, the method comprising administering the composition as disclosed herein, the pharmaceutical composition as disclosed herein, or the lyophilized composition as disclosed herein to the subject in need thereof.

In one embodiment, the disease or disorder is a disease or disorder of the gastrointestinal tract. In one embodiment, the disease or disorder is Crohn's disease, irritable bowel syndrome, ulcerative colitis or gastrointestinal cancer.

Preferable therapeutic agents include nucleotides, which can be RNA or DNA. Particularly preferred therapeutic agents comprise short interfering RNA segments, which can reduce or eliminate the production of harmful proteins in the gastro-intestinal system. The RNA fragments to be included in the LNPs will be determined by the treatment desired for the patients under consideration. For example, siRNAs designed to silence genes which produce irritant proteins for a variety of disease states are known. Other lipids and formulations embodying the teachings of the present application will be producible by those of skill in the art.

In one embodiment, the lyophilized composition or the pharmaceutical composition is orally administered as a lyophilized admixture. In one embodiment, the lyophilized composition or the pharmaceutical composition are encapsulated in a capsule. In one embodiment, the capsule further comprises a pharmaceutically acceptable carrier, diluent, excipient, solubilizer, or emulsifier.

In one embodiment, the lyophilized composition or the pharmaceutical composition is intravenously administered as a lyophilized admixture.

In another aspect, the present invention provides a method of treating a disease or disorder in a subject comprising administering to the subject the composition as disclosed herein, the pharmaceutical composition as disclosed herein, or the lyophilized composition as disclosed herein reconstituted in a liquid medium.

In one embodiment, the reconstituted lyophilized composition is administered orally. In one embodiment, the reconstituted lyophilized composition is administered intravenously. In one embodiment, the reconstituted lyophilized composition is administered subcutaneously or intramuscularly.

### BRIEF DESCRIPTION OF THE DRAWINGS

FIGS. 1A-1D. FIG. 1A depicts the samples before and after lyophilization. FIG. 1B depicts P/LNP=1.18 with 5% sucrose samples after lyophilization. FIG. 1C depicts a size histogram before and after lyophilization. Size is analyzed after being diluted 100× in deionized water (DIW). Each sample is analyzed 3 times. The error bars depict the standard deviation. FIG. 1D depicts a size histogram before and after lyophilization. Size is analyzed after being diluted 100× in DIW. Each sample is analyzed 3 times. The error bars depict the standard deviation.

FIG. 2 depicts size distribution before and after lyophilization for the formulations B, C, D, E1, E4, F, and G. Size was analyzed after being diluted 100× in DIW. Each sample is analyzed 3 times. The error bars depict the standard deviation.

FIGS. 3A and 3B. FIG. 3A Size distribution for 50 mM HEPES dialyzed LNP-S100 formulations with 40% sucrose and without sucrose content while undergoing lyophilization. Size was analyzed after being diluted 100× in DIW. Each sample was analyzed 3 times. The error bars depict the standard deviation. FIG. 3B Size distribution for 50 mM HEPES dialyzed LNP-S100 formulations with different Sucrose content while lyophilization. Size was analyzed after being diluted 100× in DIW. Each sample was analyzed 3 times. The error bars depict the standard deviation.

FIG. 4 depicts size distribution for 1×PBS dialyzed LNP-S100 formulations with different Sucrose content while lyophilization. Size was analyzed after being diluted 100× in DIW. Each sample was analyzed 3 times. The error bars depict the standard deviation.

FIG. 5A RAW264.7 cells treated with reconstituted lyophilized LNPs (A, B, C, D, E1, E4, F, G), compared with non-lyophilized formulation LNP dialyzed against 50 mM HEPES. 5 wells per sample were analyzed and the standard deviation is depicted as error bars. FIG. 5B RAW264.7 cells treated with reconstituted lyophilized LNPs compared with non-lyophilized formulation LNP dialyzed against 1×PBS. 5 wells per sample are analyzed and the standard deviation is depicted as error bars.

FIGS. 6A and 6B: HeLa cells treated with reconstituted lyophilized LNPs compared with non-lyophilized formulation LNP dialyzed against FIG. 6A. 50 mM HEPES and FIG. 6B. 1×PBS. 5 wells per sample were analyzed, and the standard deviation is depicted as error bars.

FIGS. 7A and 7B: Comparison between LNP and PCLNP before and after lyophilization for FIG. 7A. the physico-chemical characteristics, and FIG. 7B. Efficacy.

FIGS. 8A, 8B and 8C: Retention efficacy of the LNPs and PCLNPs after subjecting them to biorelevant media. FIG. 8A shows LNP stability in Fasted Stimulated Gastric Fluid (FaSSGF) and in Fasted Stimulated Intestinal Fluid (FaSSIF) for 1 h at 37° C., testing size and ZP; encapsulation efficiency, and average factor change. FIG. 8B. Shows the phisycochenical properties and in vitro efficacy when the PCLNPs after lyophilization are incubated with FaSSGF and FaSSIF. FIG. 8C shows the retention of potency of the LNPs and PCLNPs after lyophilization, after being incubated with mucin.

FIG. 9: in vivo efficacy to knock down endogenous HPRT by lyophilized LNPs encompassing siHPRT.

FIG. 10: in vivo efficacy of expression of mRNA by lyophilized LNPs encompassing FLuc mRNA.

FIG. 11: siRNA (siTNF) quantification data in DSS induced colitis model after 5 days of dosing of siRNA LNP in a 12-day DSS model.

### DETAILED DESCRIPTION

### Definitions

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

The terms “purify,” “purified,” “purification” means to make substantially pure or clear from unwanted components, material defilement, admixture or imperfection.

The terms “significant” or “significantly” are used synonymously with the term “substantially.” The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “stable” refers to a compound that is sufficiently robust to survive isolation to a useful degree of purity from a reaction mixture, and preferably capable of formulation into an efficacious therapeutic agent. The terms “stabilize” and “stabilized,” means to make or become stable.

### Polymer Coated Lipid Nanoparticle

The present invention provides new compositions of matter, comprising at least one lipid nanoparticle encapsulating an active ingredient and an aqueous solution comprising a salt and an anionic polymer. Preferably, the salt and the anionic polymer are dissolved in the aqueous solution. In some embodiments, the composition comprises at least one lipid nanoparticle encapsulating an active ingredient and an aqueous solution comprising a salt and a polymer, wherein the salt and the polymer are dissolved in the aqueous solution. In some embodiments, the polymer is a cationic polymer or an anionic polymer.

As used herein, the “polymer coated lipid nanoparticle” or “PCLNP”, refers to at least one polymer electrostatically bound to the surface of the lipid nanoparticle forming a coat around the LNP. As used herein “electrostatically bonded”, or any grammatical derivations thereof, means bonded based on the attraction of opposite charges. For example, the polymer preferably has the opposite charge as the lipid nanoparticle, such as the polymer comprises a negative charge and the lipid nanoparticle comprises a positive charge. In some embodiments, the polymer comprises a positive charge and the lipid nanoparticle comprises a negative charge.

In some embodiments, the polymer coat covers at least 10%, at least 20%, at least 30%, at least 40%, at least 50%, at least 60%, at least 70%, at least 80%, at least 90%, at least 95%, at least 98%, or at least 100% of the surface of the lipid nanoparticle. In some embodiments, the polymer coat covers between about 5% to about 100%, about 5% to about 95%, about 5% to about 90%, about 5% to about 80%, about 5% to about 70%, about 5% to about 60%, about 5% to about 50%, about 5% to about 40%, about 5% to about 30%, about 5% to about 20%, about 5% to about 10%, about 10% to about 100%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 95%, about 20% to about 90%, about 20% to about 80%, about 20% to about 70%, about 20% to about 60%, about 20% to about 50%, about 20% to about 40%, about 20% to about 30%, about 30% to about 100%, about 30% to about 95%, about 30% to about 90%, about 30% to about 80%, about 30% to about 70%, about 30% to about 60%, about 30% to about 50%, about 30% to about 40%, about 40% to about 100%, about 40% to about 95%, about 40% to about 90%, about 40% to about 80%, about 40% to about 70%, about 40% to about 60%, about 40% to about 50%, about 50% to about 100%, about 50% to about 95%, about 50% to about 90%, about 50% to about 80%, about 50% to about 70%, about 50% to about 60%, about 60% to about 100%, about 60% to about 95%, about 60% to about 90%, about 60% to about 80%, about 60% to about 70%, about 70% to about 100%, about 70% to about 95%, about 70% to about 90%, about 70% to about 80%, about 80% to about 100%, about 80% to about 95%, about 80% to about 90%, about 90% to about 100%, about 90% to about 95%, about 95% to about 100% of the surface of the lipid nanoparticle.

In some embodiments, the polymer is an anionic polymer. In some embodiments, the anionic polymer is a methacrylic acid polymer (e.g., Eudragit® S100 (an anionic copolymer derived from methacrylic acid and methyl methacrylate (1:2)), S12,5, and L100). In some embodiments, the methacrylic acid polymer is Eudragit® S100. In some embodiments, the anionic polymer is Eudragit® L-55. In some embodiments, the polymer is poly(beta-amino ester)s (PBAEs). In some embodiments, the anionic polymer is poly(methyl methacrylate) (PMMA). In some embodiments, the anionic polymer is an acrylate, a methacrylate, a cyanoacrylate, or a nitroacrylate. In some embodiments, the anionic polymer is poly(alkyl cyanoacrylates) (PACA) or poly(butyl cyanoacrylate) (PBCA). In some embodiments, the anionic polymer is hyaluronic acid.

In some embodiments, the polymer is a cationic polymer. In some embodiments, the cationic polymer is chitosan. In some embodiments, the cationic polymer is polyethylenimine (PEI). In some embodiments, the cationic polymer is poly(L-lysine). In some embodiments, the cationic polymer is poly(acrylic acid) (PAA). In some embodiments, the cationic polymer is gelatin, cationic cellulose, cationic dextran, poly-L-ornithine (PLO), poly-L-arginine (PLR), or polyamidoamine (PAMAM).

As used herein, the phrase “N/P ratio” refers to the ratio of positively-chargeable lipid, lipidoid or polymer amine (N=nitrogen) groups to negatively-charged nucleic acid phosphate (P) groups, is perhaps one of the most important physicochemical properties of polymer-based gene delivery vehicles. In some embodiments, the nucleic acid is RNA. In some embodiments, the nucleic acid is DNA. In some embodiments, the N/P ratio is about 1 to about 35, about 1 to about 30, about 1 to about 25, about 1 to about 20, about 1 to about 15, about 1 to about 10, about 1 to about 5, about 5 to about 35, about 5 to about 30, about 5 to about 25, about 5 to about 20, about 5 to about 15, about 5 to about 10, about 10 to about 35, about 10 to about 30, about 10 to about 25, about 10 to about 20, about 10 to about 15, about 15 to about 35, about 15 to about 30, about 15 to about 25, about 15 to about 20, about 20 to about 35, about 20 to about 30, about 20 to about 25, about 25 to about 35, about 25 to about 30, or about 30 to about 35. Preferably, the nucleic acid is RNA and the N/P ratio is about 5 to about 30. In some embodiments, the nucleic acid is DNA and the N/P ratio is about 15 to about 20.

As used herein, the terms “aqueous solution comprising a salt” and “buffer” are interchangeable. In some embodiments, the aqueous solution comprising a salt is a buffer selected from the group consisting of HEPES (4-(2-hydroxyethyl)-1-piperazineethanesulfonic acid), PBS (phosphate buffered saline), TRIS (tris(hydroxymethyl)aminomethane), MES (2-(N-morpholino)ethanesulfonic acid), and citrate buffer. In one embodiment, the aqueous solution is HEPES. In one embodiment, the aqueous solution is PBS.

In some embodiments, the aqueous solution comprising a salt has a pH of about pH 7.0 to about pH 8.0. In some embodiments, the pH is about 7.6.

In some embodiments, the PCLNP composition further comprises a sugar. In some embodiments, the sugar is a disaccharide. In some embodiments, the sugar is selected from the group consisting of lactose, sucrose, trehalose, and a combination thereof.

In some embodiments, the amount of sugar in the PCLNP composition is between about 0% and about 40%. In one embodiment, the amount of sugar is between about 0.1% and about 40%, about 1% to about 40%, about 1% to about 35%, about 1% to about 30%, about 1% to about 25%, about 1% to about 20%, about 1% to about 15%, about 1% to about 10%, about 1% to about 5%, about 5% to about 40%, about 5% to about 35%, about 5% to about 30%, about 5% to about 25%, about 5% to about 20%, about 5% to about 15%, about 5% to about 10%, about 10% to about 40%, about 10% to about 35%, about 10% to about 30%, about 10% to about 25%, about 10% to about 20%, about 10% to about 15%, about 15% to about 40%, about 15% to about 35%, about 15% to about 30%, about 15% to about 25%, about 15% to about 20%, about 20% to about 40%, about 20% to about 35%, about 20% to about 30%, about 20% to about 25%, about 25% to about 40%, about 25% to about 35%, about 25% to about 30%, about 30% to about 40%, about 30% to about 35%, or about 35% to about 40%. In one embodiment, the amount of sugar is at least 5%, at least 10%, at least 20%, or at least 30%. In one embodiment, preferably the amount of sugar is about 5%.

As used herein, the term “zeta potential” refers to the electrostatic potential of a molecule or particle such as a LNP, measured at the plane of hydrodynamic slippage outside the surface of the molecule or particle. Usually measured by electrophoretic mobility. Related to the surface potential and a measure of the electrostatic forces of repulsion the particle is likely to meet when encountering another of the same sign of charge. In some embodiments, the zeta potential of the lipid nanoparticle, with or without the polymer coating, can be measured.

In some embodiments, the composition comprises a PCLNP coated with an anionic polymer. In some embodiments, the zeta potential is negative.

In some embodiments, the zeta potential is between about −0.1 mV to about −50 mV, about-0.5 mV to about −50 mV, about −1.0 mV to about −50 mV, about −2.0 mV to about −50 mV, about −5.0 mV to about −50 mV, about −10 mV to about −50 mV, about −15 mV to about −50 mV, about −20 mV to about −50 mV, about −25 mV to about −50 mV, about −30 mV to about −50 mV, about −35 mV to about −50 mV, about −40 mV to about −50 mV, about −45 mV to about −50 mV, about −0.1 mV to about −45 mV, about −0.5 mV to about −45 mV, about −1.0 mV to about −45 mV, about −2.0 mV to about −45 mV, about −5.0 mV to about −45 mV, about −10 mV to about −45 mV, about −15 mV to about −45 mV, about −20 mV to about −45 mV, about −25 mV to about −45 mV, about −30 mV to about −45 mV, about −35 mV to about −45 mV, about −40 mV to about −45 mV, about −0.1 mV to about −40 mV, about −0.5 mV to about −40 mV, about −1.0 mV to about −40 mV, about −2.0 mV to about −40 mV, about −5.0 mV to about −40 mV, about −10 mV to about −40 mV, about −15 mV to about −40 mV, about −20 mV to about −40 mV, about −25 mV to about −40 mV, about −30 mV to about −40 mV, about −35 mV to about −40 mV, about −0.1 mV to about −35 mV, about −0.5 mV to about −35 mV, about −1.0 mV to about −35 mV, about −2.0 mV to about −35 mV, about −5.0 mV to about −35 mV, about −10 mV to about −35 mV, about −15 mV to about −35 mV, about −20 mV to about −35 mV, about −25 mV to about −35 mV, about −30 mV to about −35 mV, about −0.1 mV to about −30 mV, about −0.5 mV to about −30 mV, about −1.0 mV to about −30 mV, about −2.0 mV to about −30 mV, about −5.0 mV to about −30 mV, about −10 mV to about −30 mV, about −15 mV to about −30 mV, about −20 mV to about −30 mV, about −25 mV to about −30 mV, about −0.1 mV to about −25 mV, about −0.5 mV to about −25 mV, about −1.0 mV to about −25 mV, about −2.0 mV to about −25 mV, about −5.0 mV to about −25 mV, about −10 mV to about −25 mV, about −15 mV to about −25 mV, about −20 mV to about −25 mV, about −0.1 mV to about −20 mV, about −0.5 mV to about −20 mV, about −1.0 mV to about −20 mV, about −2.0 mV to about −20 mV, about −5.0 mV to about −20 mV, about −10 mV to about −20 mV, about −15 mV to about −20 mV, about −0.1 mV to about −15 mV, about −0.5 mV to about −15 mV, about −1.0 mV to about −15 mV, about −2.0 mV to about −15 mV, about −5.0 mV to about −15 mV, about −10 mV to about −15 mV, about −0.1 mV to about −10 mV, about −0.5 mV to about −10 mV, about −1.0 mV to about −10 mV, about −2.0 mV to about −10 mV, about −5.0 mV to about −10 mV, about −0.1 mV to about −5.0 mV, about −0.5 mV to about −5.0 mV, about −1.0 mV to about −5.0 mV, about −2.0 mV to about −5.0 mV, about −0.1 mV to about −2.0 mV, about −0.5 mV to about −2.0 mV, about −1.0 mV to about −2.0, about −0.1 mV to about −1.0 mV, about −0.5 mV to about −1.0 mV, or about −0.1 mV to about −0.5. In some embodiments, the zeta potential is at least −0.1 mV, at least −0.5 mV, at least −1.0 mV, at least −2.0 mV, at least −5.0 mV, at least −10 mV, at least −15 mV, at least −20 mV, at least −25 mV, at least −30 mV, at least −35 mV, at least −40 mV, at least −45 mV, or at least −50 mV. In some embodiments, the zeta potential is at most −0.1 mV, at most −0.5 mV, at most −1.0 mV, at most −2.0 mV, at most −5.0 mV, at most −10 mV, at most −15 mV, at most −20 mV, at most −25 mV, at most −30 mV, at most −35 mV, at most −40 mV, at most −45 mV, or at most −50 mV.

In some embodiments, the composition comprises a PCLNP coated with a cationic polymer. In some embodiments, the zeta potential is positive.

In some embodiments, the zeta potential is between about +0.1 mV to about +50 mV, about +0.5 mV to about +50 mV, about +1.0 mV to about +50 mV, about +2.0 mV to about +50 mV, about +5.0 mV to about +50 mV, about +10 mV to about +50 mV, about +15 mV to about +50 mV, about +20 mV to about +50 mV, about +25 mV to about +50 mV, about +30 mV to about +50 mV, about +35 mV to about +50 mV, about +40 mV to about +50 mV, about +45 mV to about +50 mV, about +0.1 mV to about +45 mV, about +0.5 mV to about +45 mV, about +1.0 mV to about +45 mV, about +2.0 mV to about +45 mV, about +5.0 mV to about +45 mV, about +10 mV to about +45 mV, about +15 mV to about +45 mV, about +20 mV to about +45 mV, about +25 mV to about +45 mV, about +30 mV to about +45 mV, about +35 mV to about +45 mV, about +40 mV to about +45 mV, about +0.1 mV to about +40 mV, about +0.5 mV to about +40 mV, about +1.0 mV to about +40 mV, about +2.0 mV to about +40 mV, about +5.0 mV to about +40 mV, about +10 mV to about +40 mV, about +15 mV to about +40 mV, about +20 mV to about +40 mV, about +25 mV to about +40 mV, about +30 mV to about +40 mV, about +35 mV to about +40 mV, about +0.1 mV to about +35 mV, about +0.5 mV to about +35 mV, about +1.0 mV to about +35 mV, about +2.0 mV to about +35 mV, about +5.0 mV to about +35 mV, about +10 mV to about +35 mV, about +15 mV to about +35 mV, about +20 mV to about +35 mV, about +25 mV to about +35 mV, about +30 mV to about +35 mV, about +0.1 mV to about +30 mV, about +0.5 mV to about +30 mV, about +1.0 mV to about +30 mV, about +2.0 mV to about +30 mV, about +5.0 mV to about +30 mV, about +10 mV to about +30 mV, about +15 mV to about +30 mV, about +20 mV to about +30 mV, about +25 mV to about +30 mV, about +0.1 mV to about +25 mV, about +0.5 mV to about +25 mV, about +1.0 mV to about +25 mV, about +2.0 mV to about +25 mV, about +5.0 mV to about +25 mV, about +10 mV to about +25 mV, about +15 mV to about +25 mV, about +20 mV to about +25 mV, about +0.1 mV to about +20 mV, about +0.5 mV to about +20 mV, about +1.0 mV to about +20 mV, about +2.0 mV to about +20 mV, about +5.0 mV to about +20 mV, about +10 mV to about +20 mV, about +15 mV to about +20 mV, about +0.1 mV to about +15 mV, about +0.5 mV to about +15 mV, about +1.0 mV to about +15 mV, about +2.0 mV to about +15 mV, about +5.0 mV to about +15 mV, about +10 mV to about +15 mV, about +0.1 mV to about +10 mV, about +0.5 mV to about +10 mV, about +1.0 mV to about +10 mV, about +2.0 mV to about +10 mV, about +5.0 mV to about +10 mV, about +0.1 mV to about +5.0 mV, about +0.5 mV to about +5.0 mV, about +1.0 mV to about +5.0 mV, about +2.0 mV to about +5.0 mV, about +0.1 mV to about +2.0 mV, about +0.5 mV to about +2.0 mV, about +1.0 mV to about +2.0, about +0.1 mV to about +1.0 mV, about +0.5 mV to about +1.0 mV, or about +0.1 mV to about +0.5. In some embodiments, the zeta potential is at least +0.1 mV, at least +0.5 mV, at least +1.0 mV, at least +2.0 mV, at least +5.0 mV, at least +10 mV, at least +15 mV, at least +20 mV, at least +25 mV, at least +30 mV, at least +35 mV, at least +40 mV, at least +45 mV, or at least +50 mV. In some embodiments, the zeta potential is at most +0.1 mV, at most +0.5 mV, at most +1.0 mV, at most +2.0 mV, at most +5.0 mV, at most +10 mV, at most +15 mV, at most +20 mV, at most +25 mV, at most +30 mV, at most +35 mV, at most +40 mV, at most +45 mV, or at most +50 mV.

In some embodiments, the zeta potential of the PCLNPs are measured before and after lyophilization. Measurement after lyophilization requires the PCLNPs to be reconstituted. In some embodiments, the difference in zeta potential of the PCLNPs before and after lyophilization is calculated by the formula

$$100 \times ( ZETA POTENTIAL before lyophilization \text{} / ZETA POTENTIAL after lyophilization ) = \% difference .$$

In some embodiments, the difference in zeta potential of the PCLNPs before and after lyophilization of the PCLNPs is less than 30%, less than 25%, less than 20%, less than 15%, less than 10%, less than 5%. In some embodiments, the difference in zeta potential of the PCLNPs before and after lyophilization of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

As used herein, the term “polydispersity index (PDI)” refers to the standard deviation (o) of the particle diameter distribution divided by the mean particle diameter. PDI is used to estimate the average uniformity of a particle solution. For example, a small value, e.g., less than 0.3, indicates a narrow particle size distribution. For example, a larger PDI value corresponds to a larger size distribution in the particle sample.

In some embodiments, the polydispersity index of the PCLNPs in a composition is less than 1.0, less than 0.9, less than 0.8, less than 0.7, less than 0.6, less than 0.5, less than 0.4, less than 0.3, less than 0.2, less than 0.1. In some embodiments, the polydispersity index of the PCLNPs in a composition is between about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.0, about 0.8 to about 0.9, or about 0.9 to about 1.0.

In some embodiments, the polydispersity index of the PCLNPs is measured before and after lyophilization. Measurement after lyophilization requires the PCLNPs to be reconstituted. In some embodiments, the difference in polydispersity index of the PCLNPs before and after lyophilization is calculated by the formula

$$100 \times ( PDI before lyophilization / PDI after lyophilization ) = \% difference .$$

In some embodiments, the difference in polydispersity index of the PCLNPs before and after lyophilization of the PCLNPs is less than 30%, less than 25%, less than 20%, less than 15%, less than 10%, less than 5%. In some embodiments, the difference in polydispersity index of the PCLNPs before and after lyophilization of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

In some embodiments, the size of the PCLNPs is measured before and after lyophilization. Measurement after lyophilization requires the PCLNPs to be reconstituted. In some embodiments, the difference in size of the PCLNPs before and after lyophilization is calculated by the formula

$$100 \times ( SIZE before lyophilization / SIZE after lyophilization ) = \% difference .$$

In some embodiments, the difference in size of the PCLNPs before and after lyophilization of the PCLNPs is less than 30%, less than 25%, less than 20%, less than 15%, less than 10%, less than 5%. In some embodiments, the difference in size of the PCLNPs before and after lyophilization of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

### Lipid Nanoparticle

As used herein, the term “lipid nanoparticle” or “LNP” refers to a structure comprising a lipophilic core surrounded by a hydrophilic phase encapsulating the core. The ionic interaction resulting from the different lipophilic and hydrophilic components of the nanoparticle generates independent and observable physical characteristics, the mean size of which is equal to or less than 1 μm, i.e., a mean size comprised between 1 and 1000 nm.

“Average size” is understood as the average diameter of the population of nanoparticles comprising the lipophilic phase and the hydrophilic phase. The mean size of these systems can be measured by standard methods known by the person skilled in the art, and which are described, for example, in the experimental part below.

An “anionic lipid” refers to a lipid capable of being negatively charged. Exemplary anionic lipids include one or more phosphate group(s) which bear a negative charge, for example at physiological pHs. In some embodiments, the anionic lipid does not include a serine moiety, including phosphatidylserine lipids.

As used herein, the term “ionizable lipid” refers to lipids having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. Ionizable lipids are also referred to as cationic lipids herein.

As used herein, the term “non-cationic lipid” refers to any amphipathic lipid as well as any other neutral lipid or anionic lipid. Accordingly, the non-cationic lipid can be a neutral uncharged, zwitterionic, or anionic lipid.

As used herein, the term “conjugated lipid” refers to a lipid molecule conjugated with a non-lipid molecule, such as a PEG (e.g., PEG-lipid), polyoxazoline, polyamide, or polymer (e g., cationic polymer).

The term “neutral lipid”, also known as “helper lipid”, refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

In some embodiments, the lipid nanoparticle comprises at least one lipid selected from the group consisting of a cationic lipid, an anionic lipid, a neutral lipid, a zwitterionic lipid, an ionizable lipid, a helper lipid, a cholesterol, a PEG-lipid, phospholipid, and a combination thereof.

In some embodiments, the lipid nanoparticle comprises an ionizable lipid and a cationic lipid. In some embodiments, the combination of the ionizable lipid and the cationic lipid is more than about 20%, more than about 30%, more than about 40%, more than about 50%, or more than about 60% of the total lipid in the lipid nanoparticle. In some embodiments, the combination of the ionizable lipid and the cationic lipid is between about 20% to about 70%, about 20% to about 60%, about 20% to about 50%, about 20% to about 40%, about 20% to about 30%, about 30% to about 70%, about 30% to about 60%, about 30% to about 50%, about 30% to about 40%, about 40% to about 70%, about 40% to about 60%, about 40% to about 50%, about 50% to about 70%, about 50% to about 60%, or about 60% to about 70% of the total lipid in the lipid nanoparticle. In some embodiments, the combination of the ionizable lipid and the cationic lipid is at least 20%, at least 30%, at least 40%, at least 50%, or at least 60% of the total lipid in the lipid nanoparticle.

In some embodiments, the ionizable lipid is a primary amino lipid, a secondary amino lipid, a tertiary amino lipid, or a mulitvalent lipid. In some embodiments, the ionizable lipid is more than about 5%, more than about 10%, more than about 20%, more than about 30%, more than about 40%, or more than about 50% of the total lipid in the lipid nanoparticle. In some embodiments, the ionizable lipid is between about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 50%, about 10% to about 45%, about 10% to about 40%, about 10% to about 35%, about 10% to about 30%, about 10% to about 25%, about 10% to about 20%, about 10% to about 15%, about 15% to about 50%, about 15% to about 45%, about 15% to about 40%, about 15% to about 35%, about 15% to about 30%, about 15% to about 25%, about 15% to about 20%, about 20% to about 50%, about 20% to about 45%, about 20% to about 40%, about 20% to about 35%, about 20% to about 30%, about 20% to about 25%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, about 35% to about 40%, about 40% to about 50%, about 40% to about 45%, or about 45% to about 50% of the total lipid in the lipid nanoparticle. In some embodiments, the ionizable lipid at least 5%, at least 10%, at least 15%, at least 20%, at least 30%, at least 40%, or at least 50% of the total lipid in the lipid nanoparticle.

In some embodiments, the cationic lipid is a quaternary amino lipid. In some embodiments, the cationic lipid is more than about 5%, more than about 10%, more than about 20%, more than about 30%, more than about 40%, or more than about 50% of the total lipid in the lipid nanoparticle. In some embodiments, the cationic lipid is between about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 50%, about 10% to about 45%, about 10% to about 40%, about 10% to about 35%, about 10% to about 30%, about 10% to about 25%, about 10% to about 20%, about 10% to about 15%, about 15% to about 50%, about 15% to about 45%, about 15% to about 40%, about 15% to about 35%, about 15% to about 30%, about 15% to about 25%, about 15% to about 20%, about 20% to about 50%, about 20% to about 45%, about 20% to about 40%, about 20% to about 35%, about 20% to about 30%, about 20% to about 25%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, about 35% to about 40%, about 40% to about 50%, about 40% to about 45%, or about 45% to about 50% of the total lipid in the lipid nanoparticle. In some embodiments, the cationic lipid at least 5%, at least 10%, at least 15%, at least 20%, at least 30%, at least 40%, or at least 50% of the total lipid in the lipid nanoparticle.

In some embodiments, the ionizable lipid is C12-200. In some embodiments, the cationic lipid is 1,2-di-O-octadecenyl-3-trimethylammonium propane (DOTMA). In some embodiments, the ionizable lipid is C12-200 and the cationic lipid is 1,2-di-O-octadecenyl-3-trimethylammonium propane (DOTMA). In some embodiments, the C12-200 is about 30% of the total lipid in the lipid nanoparticle and DOTMA is about 20% of the total lipid in the lipid nanoparticle.

In some embodiments, the cationic lipid is selected from the group of consisting of 3β-[N—(N′,N′-dimethylaminoethane)-carbamoyl]cholesterol hydrochloride (DC-Cholesterol), DOTAP:DOPE (1:1 w/w), DOTAP:DOPE (1:1, w/w)+0.1 wt. % NBD-DOPE, 1-oleoyl-2-[6-[(7-nitro-2-1,3-benzoxadiazol-4-yl)amino]hexanoyl]-3-trimethylammonium propane (chloride salt) (fluorescent DOTAP), N-(2-hydroxyethyl)-N,N-dimethyl-2,3-bis(oleoyloxy) propan-1-aminium bromide (DORI), O,O′-ditetradecanoyl-N-(α-trimethylammonioacetyl) diethanolamine chloride (DC-6-14), 1,2-dilauroyl-sn-glycero-3-ethylphosphocholine (chloride salt) (12:0 EPC), 1,2-dimyristoyl-sn-glycero-3-ethylphosphocholine (chloride salt) (14:0 EPC), 1,2-dipalmitoyl-sn-glycero-3-ethylphosphocholine (chloride salt) (16:0 EPC), 1,2-distearoyl-sn-glycero-3-ethylphosphocholine (chloride salt) (18:0 EPC), 1,2-dioleoyl-sn-glycero-3-ethylphosphocholine (chloride salt) (18:1 EPC), 1-palmitoyl-2-oleoyl-sn-glycero-3-ethylphosphocholine (chloride salt) (16:0-18:0 EPC), 1,2-dimyristoleoyl-sn-glycero-3-ethylphosphocholine (Tf salt) (14:1 EPC), Dimethyldioctadecylammonium (Bromide Salt) (18:0 DDAB), 1,2-dimyristoyl-3-trimethylammonium-propane (chloride salt) (14:0 TAP), 1,2-dipalmitoyl-3-trimethylammonium-propane (chloride salt) (16:0 TAP), 1,2-stearoyl-3-trimethylammonium-propane (chloride salt) (18:0 TAP), 1,2-dioleoyl-3-trimethylammonium-propane (chloride salt) (18:1 TAP (DOTAP)), 1,2-dioleoyl-3-trimethylammonium-propane (methyl sulfate salt) (18:1 TAP (DOTAP, MS salt)), or 1,2-di-O-octadecenyl-3-trimethylammonium propane (chloride salt) (DOTMA).

In some embodiments, the LNP net charge is positive at pH 7. In some embodiments, the LNP net charge is negative at pH 7. In some embodiments, the LNP net charge is neutral at pH 7.

### Therapeutic Agents

As used herein, the term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents” or “active ingredients.” Such agents include, but are not limited to, cytotoxins, radioactive ions, chemotherapeutic agents, small molecule drugs, proteins, and nucleic acids.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

In one embodiment, the nucleic acid molecule is between about 10 nucleotides (nt) and 20 kilobases (kb), about 500 nt to about 20 kb, about 1 kb to about 20 kb, about 2 kb to about 20 kb, about 3 kb to about 20 kb, about 4 kb to about 20 kb, about 5 kb to about 20 kb, about 6 kb to about 20 kb, about 7 kb to about 20 kb, about 8 kb to about 20 kb, about 9 kb to about 20 kb, about 10 kb to about 20 kb, about 15 kb to about 20 kb, about 10 nt and 15 kb, about 500 nt to about 15 kb, about 1 kb to about 15 kb, about 2 kb to about 15 kb, about 3 kb to about 15 kb, about 4 kb to about 15 kb, about 5 kb to about 15 kb, about 6 kb to about 15 kb, about 7 kb to about 15 kb, about 8 kb to about 15 kb, about 9 kb to about 15 kb, about 10 kb to about 15 kb, about 10 nt and 10 kb, about 500 nt to about 10 kb, about 1 kb to about 10 kb, about 2 kb to about 10 kb, about 3 kb to about 10 kb, about 4 kb to about 10 kb, about 5 kb to about 10 kb, about 6 kb to about 10 kb, about 7 kb to about 10 kb, about 8 kb to about 10 kb, about 9 kb to about 10 kb, about 10 nt and 8 kb, about 500 nt to about 8 kb, about 1 kb to about 8 kb, about 2 kb to about 8 kb, about 3 kb to about 8 kb, about 4 kb to about 8 kb, about 5 kb to about 8 kb, about 6 kb to about 8 kb, about 7 kb to about 8 kb, about 10 nt and 6 kb, about 500 nt to about 6 kb, about 1 kb to about 6 kb, about 2 kb to about 6 kb, about 3 kb to about 6 kb, about 4 kb to about 6 kb, about 5 kb to about 6 kb, about 10 nt and 5 kb, about 500 nt to about 5 kb, about 1 kb to about 5 kb, about 2 kb to about 5 kb, about 3 kb to about 5 kb, about 4 kb to about 5 kb, about 10 nt and 4 kb, about 500 nt to about 4 kb, about 1 kb to about 4 kb, about 2 kb to about 4 kb, about 3 kb to about 4 kb, about 10 nt and 3 kb, about 500 nt to about 3 kb, about 1 kb to about 3 kb, about 2 kb to about 3 kb, about 10 nt and 2 kb, about 500 nt to about 2 kb, about 1 kb to about 2 kb, about 10 nt and 1 kb, about 500 nt to about 1 kb, or about 10 nt to about 500 nt. Preferably, the nucleic acid is RNA, e.g., siRNA. Preferably, the siRNA is about 20 nt to about 25 nt. Preferably, the nucleic acid is DNA, e.g., viral vector, and is about 3 kb to about 20 kb.

The nucleotide analog may be any molecule obtained by modifying a ribonucleotide, deoxyribonucleotide, RNA, or DNA to improve nuclease resistance, to stabilize, to enhance affinity with a complementary-strand nucleic acid or to enhance cell permeability as compared with RNA or DNA, or for visualization. The nucleotide analog may be a naturally-occurring molecule or a non-natural molecule, and examples thereof include a sugar-modified nucleotide analog and a phosphodiester bond-modified nucleotide analog.

The sugar-modified nucleotide analog may be any one obtained by addition of or substitution with a substance having an arbitrary chemical structure for a part or the whole of the chemical structure of the sugar of a nucleotide. Specific examples thereof include a nucleotide analog substituted with 2′-O-methylribose, a nucleotide analog substituted with 2′-O-propylribose, a nucleotide analog substituted with 2′-methoxyethoxyribose, a nucleotide analog substituted with 2′-O-methoxyethylribose, a nucleotide analog substituted with 2′-O-[2-(guanidinium)ethyl]ribose, a nucleotide analog substituted with 2′-O-fluororibose, a crosslinked artificial nucleic acid provided with two cyclic structures by introducing a crosslinked structure to the sugar moiety (Bridged Nucleic Acid) (BNA), more specifically, a locked artificial nucleic acid in which the oxygen atom at position 2′ and the carbon atom at position 4′ are crosslinked via methylene (Locked Nucleic Acid) (LNA) and an ethylene-crosslinked artificial nucleic acid (Ethylene bridged nucleic acid) (ENA).

The phosphodiester bond-modified nucleotide analog may be any one obtained by addition of or substitution with an arbitrary chemical substance for a part or the whole of the chemical structure of the phosphodiester bond of a nucleotide. Specific examples thereof include a nucleotide analog substituted with a phosphorothioate bond and a nucleotide analog substituted with an N3′-P5′ phosphoramidate bond.

The nucleic acid derivative may be any molecule obtained by adding to a nucleic acid another chemical substance to improve nuclease resistance, to stabilize, to enhance affinity with a complementary-strand nucleic acid, or to enhance cell permeability as compared with the nucleic acid, or for visualization. Specific examples thereof include a derivative with 5′-polyamine added, a derivative with cholesterol added, a derivative with steroid added, a derivative with bile acid added, a derivative with vitamin added, a derivative with Cy5 added, a derivative with Cy3 added, a derivative with 6-FAM added, and a derivative with biotin added.

The nucleic acid in the present invention is not limited to a particular nucleic acid, and may be a nucleic acid, for example, for the purpose of ameliorating a disease, a symptom, a disorder, or sickness, and mitigating a disease, a symptom, a disorder, or pathological condition or preventing the onset thereof (herein, occasionally referred to as “treatment or the like of a disease”), or a nucleic acid for regulating expression of a desired protein that is useful for research, even though the protein does not contribute to treatment or the like of a disease.

Specific examples of the nucleic acid in the present invention include siRNAs, shRNAs, miRNAs, miRNA mimics, antisense nucleic acids, ribozymes, mRNAs, decoy nucleic acids, aptamers, plasmid DNAs, Cosmid DNAs, and BAC DNAs. The nucleic acid is preferably an RNA such as an siRNA and an mRNA or an analog or derivative obtained by artificially modifying an RNA.

In the present invention, the “siRNA” refers to a double-stranded RNA or relative thereof consisting of 10 to 30 nucleotides, preferably of 15 to 25 nucleotides, and including complementary sequences. The siRNA includes protruding nucleotides preferably of one to three nucleotides, more preferably of two nucleotides, at the 3′-end. The moiety of complementary sequences may be completely complementary or include non-complementary nucleotides, but preferably are completely complementary.

The siRNA in the present invention is not limited to a particular siRNA, and, for example, an siRNA for knockdown of gene expression against a disease-related gene may be used. A disease-related gene refers to any gene or polynucleotide generating a transcription or translation product at an abnormal level or in an abnormal form in cells derived from tissue of a patient, as compared with tissue or cells from a disease-free control. For the siRNA in the present invention, an siRNA to regulate expression of a desired protein useful for research may be used.

In the present invention, the “mRNA” refers to an RNA including a nucleotide sequence that can be translated into a protein. The mRNA in the present invention is not limited to a particular mRNA and may be any mRNA capable of expressing a desired protein in cells. The mRNA is preferably an mRNA useful for pharmaceutical applications (e.g., applications of disease treatment) and/or applications for research, and examples of such mRNAs include an mRNA to express a marker protein such as luciferase in cells.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “RNA” means a molecule comprising at least one ribonucleotide residue. By “ribonucleotide” is meant a nucleotide with a hydroxyl group at the 2′ position of a β-D-ribo-furanose moiety. The terms includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an interfering RNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. As used herein, the terms “ribonucleic acid” and “RNA” refer to a molecule containing at least one ribonucleotide residue, including siRNA, antisense RNA, single stranded RNA, microRNA, mRNA, noncoding RNA, and multivalent RNA.

In some embodiments the RNA is a self-replicating RNA. In some embodiments, the RNA is mRNA. In some embodiments, the RNA is siRNA. In some embodiments, the nucleic acid is from about 1000 nucleotides to about 13000 nucleotides in length.

The lipid nanoparticles of the disclosure also typically have a total lipid: RNA ratio (mass/mass ratio) of from about 1:1 to about 100:1, from about 1:1 to about 50:1, from about 2:1 to about 45:1, from about 3:1 to about 40:1, from about 5:1 to about 38:1, or from about 6:1 to about 40:1, or from about 7:1 to about 35:1, or from about 8:1 to about 30:1; or from about 10:1 to about 25:1; or from about 8:1 to about 12:1; or from about 13:1 to about 17:1; or from about 18:1 to about 24:1; or from about 20:1 to about 30:1.

In some embodiments, the lipid nanoparticles comprise an RNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the RNA encapsulated therein.

The encapsulated siRNA is calculated by the formula: [(total siRNA-free siRNA)/(total siRNA)]×100%.

The term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

### Disease and Disorders

The disease is not limited to a particular disease, and examples of the disease include diseases listed below. Contents in each “( )” are examples of the corresponding disease-related gene, except for the case that specific disease examples are listed. Another example of the nucleic acid in the present invention is a nucleic acid that regulates the expression level of any of those disease-related genes (or a protein encoded by the disease-related gene).

(1) Diseases of blood system [anaemia (CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, ASAT), bare lymphocyte syndrome (TAPBP, TPSN, TAP2, ABCB3, PSF2, RING11, MHC2TA, C2TA, RFX5), hemorrhagic diseases (TBXA2R, P2RX1, P2X1), factor H and factor H-like 1 deficiencies (HF1, CFH, HUS), factor V and factor VIII deficiencies (MCFD2), factor VII deficiency (F7), factor X deficiency (F10), factor XI deficiency (F11), factor XII deficiency (F12, HAF), factor XIIIA deficiency (F13A1, F13A), factor XIIIB deficiency (F13B), Fanconi's anaemia (FANCA, FACA, FA1, FA, FAA, FAAP95, FAAP90, F1134064, FANCB, FANCC, FACC, BRCA2, FANCD1, FANCD2, FANCD, FACD, FAD, FACE, FACE, FANCF, XRCC9, FANCG, BRIP1, BACH1, FANCJ, PHF9, FANCL, FANCM, KIAA1596), haemophagocytic lymphohistiocytosis (PRF1, HPLH2, UNC13D, MUNC13-4, HPLH3, HLH3, FHL3), haemophilia A (F8, F8C, HEMA), haemophilia B (F9, HEMB), haemorrhagic disorders (PI, ATT, F5), leucocyte deficiency (ITGB2, CD18, LCAMB, LAD, EIF2B1, EIF2BA, EIF2B2, EIF2B3, EIF2B5, LVWM, CACH, CLE, EIF2B4), sickle cell anaemia (HBB), thalassaemia (HBA2, HBB, HBD, LCRB, HBA1), and so on];

(2) inflammatory/immunologic diseases [AIDS (KIR3DL1, NKAT3, NKB1, AMB11, KIR3DS1, IFNG, CXCL12, SDF1), autoimmune lymphoproliferative syndrome (TNFRSF6, APT1, FAS, CD95, ALPS1A), combined immunodeficiency (IL2RG, SCIDX1, SCIDX, IMD4), HIV infection (CCL5, SCYA5, D17S135E, TCP228, IL10, CSIF, CMKBR2, CCR2, DMKBR5, CCCKR5, CCR5), immunodeficiency (CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, TACI), inflammation (IL10, IL-1, IL-13, IL-17, IL-23, CTLA4), severe combined immunodeficiency (JAK3, JAKL, DCLREIC, ATREMIS, SCIDA, RAG1, RAG2, ADA, PTPRC, CD45, LCA, IL7R, CD3D, T3D, IL2RG, SCIDX1, SCIDX, IMD4), rheumatoid arthritis, psoriasis, inflammatory bowel diseases (e.g., Crohn's disease, ulcerative colitis), Sjogren's syndrome, Behcet's disease, multiple sclerosis, systemic lupus erythematosus, lupus nephritis, discoid lupus erythematosus, Castleman's disease, ankylosing spondylitis, polymyositis, dermatomyositis, polyarteritis nodosa, mixed connective tissue disease, dermatosclerosis, lupus profundus, chronic thyroiditis, Graves' disease, autoimmune gastritis, type I and type II diabetes mellitus, autoimmune haemolytic anaemia, autoimmune neutropenia, thrombocytopenia, atopic dermatitis, chronic active hepatitis, myasthenia gravis, graft-versus-host disease, Addison's disease, abnormal immune response, arthritis, dermatitis, radiodermatitis, primary biliary cirrhosis, and so on];

(3) metabolic/liver/kidney diseases [amyloid neuropathy (TTR, PALB), amyloidosis (APOA1, APP, AAA, CVAP, AD1, GSN, FGA, LYZ, TTR, PALB), non-alcoholic steatohepatitis and hepatic fibrosis (COL1A1), cirrhosis (KRT18, KRT8, CIRHIA, NAIC, TEX292, KIAA1988), cystic fibrosis (CFTR, ABCC7, CF, MRP7), glycogen storage disease (SLC2A2, GLUT2, G6PC, G6PT, G6PT1, GAA, LAMP2, LAMPB, AGL, GDE, GBE1, GYS2, PYGL, PFKM), hepatocellular adenoma (TCF1, HFN1A, MODY3), hepatic failure (SCOD1, SCO1), hepatic lipase deficiency (LIPC), hepatoblastoma (CTNNB1, PDFGRL, PDGRL, PRLTS, AXIN1, AXIN, TP53, P53, LFS1, IGF2R, MPRI, MET, CASP8, MCH5), medullary cystic kidney disease (UMOD, HNFJ, FJHN, MCKD2, ADMCKD2), phenylketonuria (PAH, PKU1, QDPR, DHPR, PTS), multicystic kidney and liver diseases (FCYT, PKHD1, APRKD, PDK1, PDK2, PDK4, PDKTS, PRKCSH, G19P1, PCLD, SEC63), and so on];

(4) nervous system diseases [ALS (SOD1, ALS2, STEX, FUS, TARDBP, VEGF), Alzheimer's disease (APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, AD3), autism (BZRAP1, MDGA2, GLO1, MECP2, RTT, PPMX, MRX16, MRX79, NLGN3, NLGN4, KIAA1260, AUTSX2), fragile X syndrome (FMR2, FXR1, FXR2, mGLUR5), Huntington's disease (HD, IT15, PRNP, PRIP, JPH3, JP3, HDL2, TBP, SCA17), Parkinson's disease (NR4A2, NURR1, NOT, TINUR, SNCAIP, TBP, SCA17, SNCA, NACP, PARK1, PARK4, DJ1, DBH, NDUFV2), Rett syndrome (MECP2, RTT, PPMX, MRX16, MRX79, CDKL5, STK9), schizophrenia (GSK3,5-HTT, COMT, DRD, SLC6A3, DAOA, DTNBP1), secretase-related disorder (APH-1), and so on];

(5) eye diseases [age-related macular degeneration (Abcr, Cc12, cp, Timp3, cathepsin D, Vldlr, Ccr2), cataract (CRYAA, CRYA1, CRYBB2, CRYB2, PITX3, BFSP2, CP49, CP47, PAX6, AN2, MGDA, CRYBA1, CRYB1, CRYGC, CRYG3, CCL, LIM2, MP19, CRYGD, CRYG4, BSFP2, CP49, CP47, HSF4, CTM, MW, AQPO, CRYAB, CRYA2, CTPP2, CRYBB1, CRYGD, CRYG4, CRYA1, GJA8, CX50, CAE1, GJA3, CX46, CZP3, CAE3, CCM1, CAM, KRIT1), corneal opacity (APOA1, TGFB1, CSD2, CDGG1, CSD, BIGH3, CDG2, TASTD2, TROP2, M1S1, VSX1, RINX, PPCD, PPD, KTCN, COL8A2, FECD, PPCD2, PIP5K3, CFD), congenital hereditary corneal plana (KERA, CNA2), glaucoma (MYOC, TIGR, GLC1A, JOAG, GPOA, OPTN, GLCIE, FIP2, HYPL, NRP, CYP1B1, GLC3A, OPA1, NTG, NPG, CYP1B1, GLC3A), Leber's congenital amaurosis (CRB1, RP12, CRX, CORD2, CRD, RPGRIP1, LCA6, CORDS, RPE65, RP20, AIPL1, LCA4, GUCY2D, GUC2D, LCA1, CORD6, RDH12, LCA3), macular dystrophy (ELOVL4, ADMD, STGD2, STGD3, RDS, RP7, PRPH2, PRPH, AVMD, AOFMD, VMD2), and so on]; and

(6) neoplastic diseases [malignant tumor, neovascular glaucoma, infantile hemangioma, multiple myeloma, chronic sarcoma, metastatic melanoma, Kaposi's sarcoma, angioproliferation, cachexia, metastasis or the like of breast cancer, cancer (e.g., colorectal cancer (e.g., familial colorectal cancer, hereditary nonpolyposis colorectal cancer, gastrointestinal stromal tumor), lung cancer (e.g., non-small cell lung cancer, small cell lung cancer, malignant mesothelioma), mesothelioma, pancreatic cancer (e.g., pancreatic ductal carcinoma), gastric cancer (e.g., papillary adenocarcinoma, mucinous adenocarcinoma, adenosquamous cell carcinoma), breast cancer (e.g., invasive ductal breast carcinoma, noninvasive ductal breast carcinoma, inflammatory breast carcinoma), ovarian cancer (e.g., epithelial ovarian cancer, extragonadal germ cell tumor, ovarian germ cell tumor, low-grade ovarian tumor), prostate cancer (e.g., hormone-dependent prostate cancer, hormone-independent prostate cancer), liver cancer (e.g., primary liver cancer, extrahepatic bile duct cancer), thyroid cancer (e.g., medullary thyroid cancer), kidney cancer (e.g., renal cell carcinoma, transitional cell carcinoma of renal pelvis and ureter), uterine cancer, brain tumor (e.g., pineal astrocytoma, pilocytic astrocytoma, diffuse astrocytoma, anaplastic astrocytoma), melanoma, sarcoma, bladder cancer, hematological cancer or the like including multiple myeloma, pituitary adenoma, glioma, acoustic neurinoma, retinal sarcoma, pharyngeal cancer, laryngeal cancer, tongue cancer, thymoma, esophageal carcinoma, duodenal cancer, colon cancer, rectal cancer, hepatocellular carcinoma, pancreatic endocrine tumor, bile duct cancer, gallbladder cancer, penile cancer, ureteric cancer, testicular tumor, vulvar carcinoma, cervix cancer, corpus uteri carcinoma, uterine sarcoma, trophoblastic disease, vaginal cancer, skin cancer, mycosis fungoides, basalioma, soft part sarcoma, malignant lymphoma, Hodgkin's disease, myelodysplastic syndrome, adult T-cell leukemia, chronic myeloproliferative disease, pancreatic endocrine tumor, fibrous histiocytoma, leiomyosarcoma, rhabdomyosarcoma, carcinoma of unknown primary), leukaemia (e.g., acute leukaemia (e.g., acute lymphocytic leukaemia, acute myeloid leukaemia), chronic leukaemia (e.g., chronic lymphocytic leukaemia, chronic myeloid leukaemia), myelodysplastic syndrome), uterine sarcoma (e.g., uterine mesodermal mixed tumor, uterine leiomyosarcoma, endometrial stromal tumor), myelofibrosis, and so on].

### Pharmaceutical Compositions

The composition of the present invention as a drug may be produced by using a method as disclosed herein with a pharmaceutically acceptable carrier. Examples of the dosage form of the drug include formulations for oral administration (e.g., a capsule, tablet, or liquid) blended with a conventional auxiliary such as a buffering agent and/or a stabilizer. Examples of the dosage form of the drug include formulations for parenteral administration (e.g., a liquid such an injection) blended with a conventional auxiliary such as a buffering agent and/or a stabilizer. Examples of the dosage form of the drug include formulations for topical administration, such as an ointment, a cream, a liquid, and a plaster, blended with a conventional pharmaceutical carrier.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable excipient,” refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The composition of the present invention may be used for introduction of an active ingredient into various types of cells, tissues, or organs. Examples of cells to which the composition of the present invention may be applied include mesenchymal stem cells, neural stem cells, skin stem cells, splenocytes, nerve cells, glial cells, pancreatic B cells, bone marrow cells, mesangial cells, Langerhans cells, epidermal cells, epithelial cells, endothelial cells, fibroblasts, fiber cells, muscle cells (e.g., skeletal muscle cells, cardiac muscle cells, myoblasts, muscle satellite cells, smooth muscle cells), fat cells, blood cells (e.g., macrophages, T cells, B cells, natural killer cells, mast cells, leukocytes, neutrophils, basophils, eosinophils, monocytes, megakaryocytes, hematopoietic stem cells), synoviocytes, chondrocytes, osteocytes, osteoblasts, osteoclasts, mammary cells, hepatocytes or stromal cells, ova, spermatids, or precursor cells capable of inducing differentiation into these cells, stem cells (e.g., including induced pluripotent stem cells (iPS cells), embryonic stem cells (ES cells)), primordial germ cells, oocytes, and fertilized ova. Examples of tissues or organs to which the composition of the present invention may be applied include all tissues or organs in which the above cells are present, for example, brain, sites of brain (e.g., olfactory bulb, amygdala, basal ganglion, hippocampus, thalamus, hypothalamus, subthalamic nucleus, cerebral cortex, medulla oblongata, cerebellum, occipital lobe, frontal lobe, temporal lobe, putamen, caudate nucleus, callosum, substantia nigra), spinal cord, pituitary gland, stomach, pancreas, kidney, liver, gonad, thyroid, gallbladder, bone marrow, adrenal gland, skin, lung, digestive tract (e.g., large intestine, small intestine), blood vessel, heart, thymus, spleen, submandibular gland, peripheral blood, peripheral blood cells, prostate, placenta, uterus, bones, joints, and muscles (e.g., skeletal muscle, smooth muscle, cardiac muscle).

The composition of the present invention is particularly superior in efficiency to introduce a nucleic acid into target cells.

The compound, the lipid particle, and the composition of the present invention are stable and have low toxicity and can be safely used. In using the composition of the present invention in vivo, or using the composition as a drug, the composition is suitably administered to a subject (e.g., a human or a non-human mammal (preferably, a human)) so that an effective amount of the nucleic acid can be delivered to targeted cells.

In using the composition of the present invention in vivo, or using the composition as a drug, the composition can be orally or parenterally (e.g., local, rectal, or intravenous administration) administered in a safe manner in the form of a pharmaceutical formulation such as tablets (including sugar-coated tablets, film-coated tablets, sublingual tablets, orally disintegrating tablets), powders, granules, capsules (including soft capsules, microcapsules), liquids, troches, syrups, emulsions, suspensions, injections (e.g., subcutaneous injections, intravenous injections, intramuscular injections, intraperitoneal injections), topical formulations (e.g., formulations for nasal administration, transdermal formulations, ointments), suppositories (e.g., rectal suppositories, vaginal suppositories), pellets, nasal formulations, pulmonary formulations (inhalations), and infusions. Each formulation may be a controlled-release formulation such as a fast-release formulation and a sustained-release formulation (e.g., a sustained-release microcapsule).

An “effective amount” or “therapeutically effective amount” of an active agent or therapeutic agent such as a therapeutic nucleic acid is an amount sufficient to produce the desired effect, e.g. an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as siRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

### Methods of Producing PCLNP

The composition of the present invention provides methods of producing a pharmaceutical composition, such as polymer coated lipid nanoparticles, as disclosed herein. In some embodiments, the lipid nanoparticles are produced from at least one lipid and at least one nucleic acid molecule and mixed with an anionic polymer dissolved in an aqueous solution comprising a salt, thereby forming PCLNPs. In some embodiments, the PCLNPs are further mixed with a pharmaceutically acceptable carrier. In some embodiments, the mixture of the PCLNPs and pharmaceutically acceptable carrier are lyophilized. In some embodiments, the anionic polymer is a methacrylic acid polymer.

In another embodiment, the present disclosure provides a method of stabilizing a lipid nanoparticle for lyophilization, where the lipid nanoparticles are produced from at least one lipid and at least one nucleic acid molecule and mixed with an anionic polymer dissolved in an aqueous solution comprising a salt, thereby forming PCLNPs. In some embodiments, the PCLNPs are lyophilized and thereby stabilized.

In another embodiment, the present disclosure provides a method of manufacturing a stable pharmaceutical composition, an anionic polymer is dissolved in an aqueous solution comprising a salt; and mixed with a lipid nanoparticle comprising at least one nucleic acid. In some embodiments, the PCLNPs are lyophilized and thereby stabilized.

In some embodiments, the anionic polymer is a methacrylic acid polymer. In some embodiments, the anionic polymer is dissolved in the aqueous solution comprising a salt prior to mixing with the LNPs. In some embodiments, the aqueous solution comprising a salt is selected from the group consisting of HEPES (4-(2-hydroxyethyl)-1-piperazineethanesulfonic acid), PBS (phosphate buffered saline), TRIS (tris(hydroxymethyl)aminomethane), MES (2-(N-morpholino)ethanesulfonic acid), and citrate buffer. In one embodiment, the aqueous solution is HEPES. In one embodiment, the aqueous solution is PBS. In some embodiments, the aqueous solution comprising a salt has a pH of about pH 7.0 to about pH 8.0. In some embodiments, the pH is about 7.6.

In some embodiments, the nucleic acid molecule is DNA or RNA. In some embodiments, the RNA is mRNA, microRNA (miRNA), or short interfering RNA (siRNA).

In some embodiments, the lipid nanoparticle comprises at least one lipid selected from the group consisting of a cationic lipid, an anionic lipid, a neutral lipid, a zwitterionic lipid, an ionizable lipid, a helper lipid, a cholesterol, a PEG-lipid, phospholipid, and a combination thereof.

In some embodiments, the lipid nanoparticle comprises an ionizable lipid and a cationic lipid. In some embodiments, the molecular ratio of the combination of the ionizable lipid and a the cationic lipid in the lipid nanoparticle is more than about 20%, more than about 30%, more than about 40%, more than about 50%, or more than about 60% of the total lipid in the lipid nanoparticle.

In some embodiments, the ionizable lipid is a primary amino lipid, a secondary amino lipid, a tertiary amino lipid, or a multivalent lipid. In some embodiments, the ionizable lipid is more than about 5%, more than about 10%, more than about 20%, more than about 30%, more than about 40%, or more than about 50% of the total lipid in the lipid nanoparticle.

In some embodiments, the cationic lipid is a quaternary amino lipid. In some embodiments, the cationic lipid is more than about 5%, more than about 10%, more than about 20%, more than about 30%, more than about 40%, or more than about 50% of the total lipid in the lipid nanoparticle.

In some embodiments, the ionizable lipid is C12-200. In some embodiments, the cationic lipid is 1,2-di-O-octadecenyl-3-trimethylammonium propane (DOTMA). In some embodiments, the ionizable lipid is C12-200 and the cationic lipid is 1,2-di-O-octadecenyl-3-trimethylammonium propane (DOTMA). In some embodiments, the C12-200 is about 30% of the total lipid in the lipid nanoparticle and DOTMA is about 20% of the total lipid in the lipid nanoparticle.

In another aspect, the present disclosure provides a method of preserving the lyophilized composition, or the pharmaceutical composition, as disclosed herein, by storing the lyophilized product at a temperature of about 2° C. to about 8° C., or at a temperature of about −20° C.

In another aspect, the present disclosure provides a method herein of reconstituting the lyophilized composition as disclosed herein by add a liquid medium to the lyophilized composition. In some embodiments, the zeta potential, size and PDI of the reconstituted PCLNPs are measured compared to the zeta potential, size and PDI of the PCLNPs prior to lyophilization and after lyophilization.

In some embodiments, the zeta potential of the PCLNPs are measured before and after reconstitution. In some embodiments, the difference in zeta potential of the PCLNPs before and after reconstitution is calculated by the formula

$$100 \times ( ZETA POTENTIAL before reconstitution \text{} / ZETA POTENTIAL after reconstitution ) = \% difference .$$

In some embodiments, the difference in zeta potential of the PCLNPs before and after reconstitution of the PCLNPs is less than 30%, less than 25%, less than 20%, less than 15%, less than 10%, less than 5%. In some embodiments, the difference in zeta potential of the PCLNPs before and after reconstitution of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

In some embodiments, the zeta potential of the PCLNPs are measured before lyophilization and after reconstitution. In some embodiments, the difference in zeta potential of the PCLNPs before lyophilization and after reconstitution is calculated by the formula

$$100 \times ( ZETA POTENTIAL before lyophilization \text{} / ZETA POTENTIAL after reconstitution ) = \% difference .$$

In some embodiments, the difference in zeta potential of the PCLNPs before lyophilization and after reconstitution of the PCLNPs is less than 30%, less than 25%, less than 20%, less than 15%, less than 10%, less than 5%. In some embodiments, the difference in zeta potential of the PCLNPs before lyophilization and after reconstitution of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

In some embodiments, the polydispersity index of the PCLNPs is measured before and after reconstitution. In some embodiments, the difference in PDI of the PCLNPs before and after reconstitution is calculated by the formula

$$100 \times ( PDI before reconstitution / PDI after reconstitution ) = \% difference .$$

In some embodiments, the difference in polydispersity index of the PCLNPs before and after reconstitution of the PCLNPs is less than 30%, less than 25%, less than 20%, less than 15%, less than 10%, less than 5%. In some embodiments, the difference in polydispersity index of the PCLNPs before and after reconstitution of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

In some embodiments, the polydispersity index of the PCLNPs is measured before lyophilization and after reconstitution. In some embodiments, the difference in PDI of the PCLNPs before lyophilization and after reconstitution is calculated by the formula

$$100 \times ( PDI before lyophilization / PDI after reconstitution ) = \% difference .$$

In some embodiments, the difference in polydispersity index of the PCLNPs before lyophilization and after reconstitution of the PCLNPs is less than 30%, less than 25%, less than 20%, less than 15%, less than 10%, less than 5%. In some embodiments, the difference in polydispersity index of the PCLNPs before lyophilization and after reconstitution of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

In some embodiments, the size of the PCLNPs is measured before and after reconstitution. In some embodiments, the difference in size of the PCLNPs before after reconstitution is calculated by the formula

$$100 \times ( SIZE before reconstitution / SIZE after reconstitution ) = \% difference .$$

In some embodiments, the difference in size of the PCLNPs before and after reconstitution of the PCLNPs is less than 30%, less than 25%, less than 20%, less than 15%, less than 10%, less than 5%. In some embodiments, the difference in size of the PCLNPs before and after reconstitution of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

In some embodiments, the size of the PCLNPs is measured before lyophilization and after reconstitution. In some embodiments, the difference in size of the PCLNPs before lyophilization and after reconstitution is calculated by the formula

$$100 \times ( SIZE before lyophilization / SIZE after reconstitution ) = \% difference .$$

In some embodiments, the difference in size of the PCLNPs before lyophilization and after reconstitution of the PCLNPs is less than 30%, less than 25%, less than 20%, less than 15%, less than 10%, less than 5%. In some embodiments, the difference in size of the PCLNPs before lyophilization and after reconstitution of the PCLNPs is about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, or about 1% to about 5%.

### Method of Treatment

The present invention provides methods of treating a subject by administering the composition as disclosed herein. The lyophilized composition, as disclosed herein, can be administered orally, intravenously, subcutaneously, intramuscularly, intraperitoneally, topically, and by infusions.

The term “patient” refers to a subject who may seek or be in need of treatment, requires treatment, is receiving treatment, will receive treatment, or a subject who is under care by a trained professional for a particular disease or condition.

The terms “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans).

The term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

### EXAMPLES

Additional embodiments of the present disclosure are illustrated in further detail in the following examples, which are not in any way intended to limit the scope of the claims.

### Example 1: Assess the Effect of the Addition of S100 into the Formulation to Retain Activity after Lyophilization

### LNP (Lipid Nano Particle) Preparation

LNPs were prepared by microfluidic mixing, where the lipid concentration was maintained at 2 mg/mL. Lipids C12-200 or MC3 or SM-102, DOTMA, Chol (Cholesterol), (DSPC) (Distearoylphosphatidylcholine, zwitterionic phospholipid), DMG-PEG (2k) (1,2-dimyristoyl-rac-glycero-3-methoxypolyethylene glycol-2000, synthetic polar lipid) is added in 30 (ionizable/cationic):20 (DOTMA):38.5 (Chol):10 (DSPC):1.5 (DMG-PEG (2k)) mol %, respectively, and the N/P (nitrogen/phosphate) ratio equals 20. siGAPDH (siRNAs directed against glyceraldehyde-3-phosphate dehydrogenase) or siHPRT (siRNAs directed against hypoxanthine-guanine phosophoribosyltransferase) was the payload used for in vitro and in vivo studies, respectively. Total Flow Rate (TFR) was set to 12 mL/min. Flow Rate Ratio (FRR) was set to 3:1 (aqueous phase: organic phase). Upon LNP preparation by microfluidic mixing, the samples were added into 20 kDa MWCO (Molecular Weight Cut Off) dialysis cassettes and dialyzed overnight against 100× vols. of 1×PBS, or 50 mM HEPES.

Routinely, the samples were diluted 100× in 1×PBS and 100-10× in 0.1×PBS to be tested for size and zeta potential, respectively using dynamic light scattering (DLS). Encapsulation was assessed using Ribogreen assay as per manufacturer's protocol. Concentration of lipid per LNP is calculated based on the siRNA concentration.

In some cases, LNPs have been concentrated using ultracentrifugation using 30 kDa MWCO membrane centrifuged at 2000G till desired concentration of siRNA is obtained. In some cases, 1×PBS dialyzed samples were buffer exchanged with 50 mM HEPES before lyophilization.

### S100 Addition

The concentration of lipid per LNP is calculated based on the siRNA concentration by back calculation based on the N/P ratio.

To avoid ionic interactions, 50 mg/mL of S100 solution was prepared in 50 mM HEPES buffer, and the pH is adjusted until the S100 completely dissolved (usually, pH=7.8). The final pH was noted.

The resulting S100 solution was added to the LNP based on the weight ratios of S100 to lipid. The sample was shaken either by rapid pipetting or vortex mixing.

Each S100-LNP formulation was assessed for size/PDI (polydispersity index) and charge, after 100× dilution in DIW (de-ionized water), and 10-50× dil in DIW, respectively.

### Lyophilization

Solutions of 10%, 20% and 40% sucrose were prepared in 50 mM HEPES buffer.

To each solution, equivolumes of respective sucrose solution was added, such that the effective concentration of sucrose in the solution is half of the stock.

Size was analyzed by 100× dilution in DIW, and Zeta potential (ZP) by 10-50× dilution in DIW after the sucrose addition step.

Samples were frozen at −80° C. overnight, followed by lyophilization on a bench lyophilizer for up to two days.

Samples were reconstituted by the addition of same quantity of DIW as the LNP volume used, or to the desired concentration required for dosing. After reconstitution, the samples were analyzed for size/PDI and charge after 100×dil in DIW, and 10-50× dil in DIW, respectively.

Total siRNA was determined based on the LNP solution that was added at the beginning. The encapsulated siRNA is calculated by the formula: [(total siRNA−free siRNA)/(total siRNA)]×100%.

### In Vitro Cell Transfection

RAW264.7 cells were plated at 10,000 cells/well in a 96 well opaque cell culture plate overnight. At the time of transfection, the media was changed to OMEM. Samples were diluted in OMEM and added to the wells. Cells were transfected for 5 hours, after which the cells were washed with 1×PBS, before being replaced by fresh media. At 48h, the media was removed, and wells were washed with 1×PBS before being lysed and analyzed using the KDAlert™ kit for GAPDH knock down and Pierce's BCA kit for total protein assay according to the manufacturer's protocol.

### Biorelevant Media Studies

FaSSGF, FaSSIF was prepared according to manufacturer's protocol. 2% and 5% Mucin was prepared in 1×PBS. For these experiments, the formulations were concentrated to mimic animal dosing (2 mg/kg of siRNA LNP). The formulation was diluted with different volumes of FaSSIF, FaSSGF and incubated at 37° C., under orbital shaking 280 rpm for 1 hour. Dilutions were made as indicated. For mucin experiments, equal volumes of LNP and Mucin was incubated at 37° C., under orbital shaking 280 rpm for 1 hour.

### Results

### LNP Preparation and Effect of Dialysis Buffer

LNPs were prepared and analyzed for size/PDI (polydispersity index), charge and encapsulation efficiency (EE) and depicted in Table 1. All formulations dialyzed against 1×PBS or 50 mM HEPES, have similar physico-chemical properties.

TABLE 1
Effect of LNP physio-chemical characteristics
when dialyzed against different buffer systems.

| Size: 100x | | | | |
|---|---|---|---|---|
| dil in 1x | | | | |
| PBS, ZP: | After LNP | | | |
| 100x dil in | are | 1x | 0.1x | 50 mM |
| 0.1x PBS | Formulated | PBS | PBS | HEPES |
| Size, nm | 61.45 ± 1.17 | 79.67 ± 1.08 | 79.33 ± 0.08 | 78.16 ± 1.55 |
| PDI | 0.03 ± 0 | 0.08 ± 0.02 | 0.1 ± 0.01 | 0.07 ± 0.02 |
| ZP, mV | 7.3 ± 0.47 | 9.43 ± 0.3 | 9.02 ± 0.81 | 6.82 ± 0.52 |
| % EE | — | 94.6 | 93.4 | 90.4 |

### S100-LNP Charge is Affected by the LNP Buffer

Charge changes are a function of the LNP buffer that is used. For instance, when an older formulation prepared in 1×PBS was used to titrate the charge on the LNP, S100 seemed to show a negative charge at as low as 0.03 (w/w) of S100/LNP (Table 2). LNPs were dialyzed against 1×PBS when titrated against increasing weight of S100 (50 mg/mL, in 50 mM HEPES). Size is measured at 100×dil in DIW, and ZP is measured at 50× dil in DIW. However, when the LNP prepared with 50 mM HEPES was used instead, the charge change observed was slow, with lower positive values observed at low S100 showing gradual neutralization of charge (Table 3).

TABLE 2
LNP dialyzed against 1x PBS when titrated against increasing
weight of S100 (50 mg/mL, in 50 mM HEPES)

| S100/LNP | Size, | | ZP, mV |
|---|---|---|---|
| (w/w) | nm | PDI | (50x dil in DIW) |
| 0.00 | 91.58 | 0.107 | 14.6 ± 1.04 |
| 0.03 | 125.8 | 0.127 | −16.2 ± 1.37 |
| 0.07 | 94.88 | 0.138 | −18.9 ± 0.624 |
| 0.17 | 90.00 | 0.117 | −22.5 ± 1.24 |
| 0.33 | 87.89 | 0.104 | −24.7 ± 0.757 |
| 1.65 | 84.68 | 0.118 | −30.2 ± 1.54 |
| 3.33 | 84.75 | 0.142 | −31.5 ± 0.551 |

TABLE 3
LNP dialyzed against 50 mM HEPES showing
gradual titration of charge with S100

| S100/LNP | ZP, mV |
|---|---|
| (w/w) | (50x dil in DIW) |
| 0.00 | 6.82 |
| 0.12 | 6.99 |
| 1.18 | −31.5 |
| 3.33 | −50.4 |

### S100 Solution Properties

To reduce differences in salt species in the system, S100 was dissolved in 1×PBS, 0.1×PBS, 50 mM HEPES, to a final concentration of 50 mg/mL. In the past when pH adjusted water was used to dissolve S100, the system crashed when added to LNP. While at the outset, it looks like S100 dissolves easily in 50 mM HEPES solution, after overnight stirring, the PBS solutions dissolved too. HEPES is lucrative given its zwitterionic property, which would ensure it doesn't interfere with the electrostatic interaction between the polymer and LNP. (Table 4)

TABLE 4
S100 Mixtures

| Solvent | 50 mM HEPES | 0.1x PBS | 1x PBS |
|---|---|---|---|
| S100, mg | 446.13 | 450.18 | 447.93 |
| Volume, mL | 8.9226 | 9.0036 | 8.9586 |
| pH at T0 | 5.85 | 2.84 | 4.79 |
| 5N NaOH | 280 | 280 | 280 |
| added, uL | | | |
| target pH | 7.8 | 7.8 | 7.8 |
| Final pH | 7.86 | 8.95 | 7.83 |
| Appearance | Clear, | Particles were | Can see some |
| | colorless, | visible at 7.81; | particles at pH = |
| | viscous | Can see | 7.83. In another |
| | | particles at | vial, when some |
| | | pH 8.95 | excess NaOH was |
| | | (see below) | added bringing the |
| | | | pH > 11, the |
| | | | solution turned |
| | | | clear |
| | | | (see below) |

### Effect of S100 on Lyophilization: 1×PBS LNP+50 mM HEPES S100

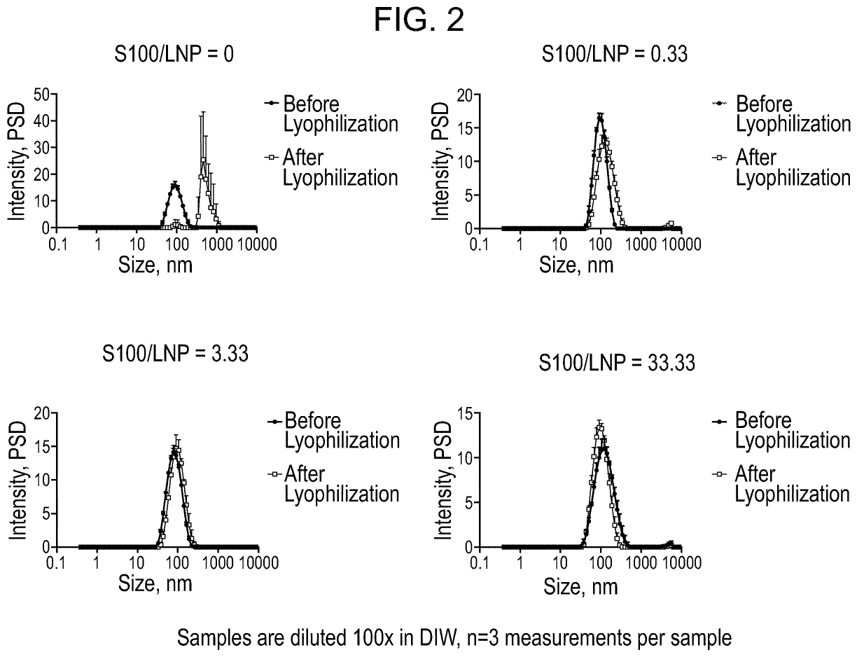

LNP dialyzed against PBS shows negative zeta potential at a lower P/LNP concentration, as observed previously. PDI for B-D are <0.2. Table 5 and FIG. 2.

TABLE 5

| Formulation: | | | |
|---|---|---|---|
| 1x PBS dialyzed | | | After |
| LNP | P/LNP (w/w) | LNP + S100 | lyophilization |
| A | 0, no Sucrose | 9.43 | 5.87 |
| B | 0.33 | −23.3 | −14.8 |
| C | 3.33 | −37.3 | −35.5 |
| D | 33.33 | −50.7 | −36.6 |

### Preliminary Study Showing S100 Modulates Lyophilization Effects on the LNP

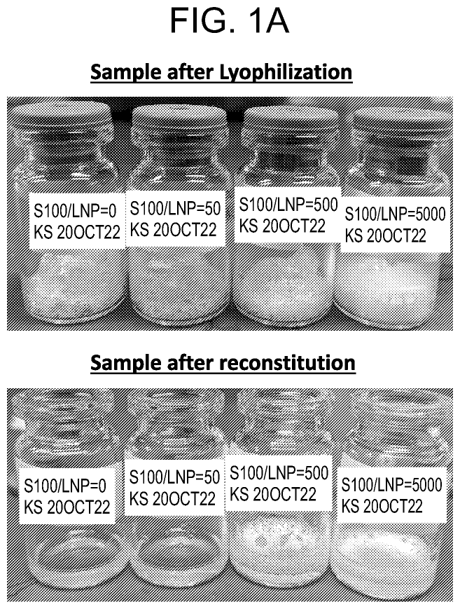

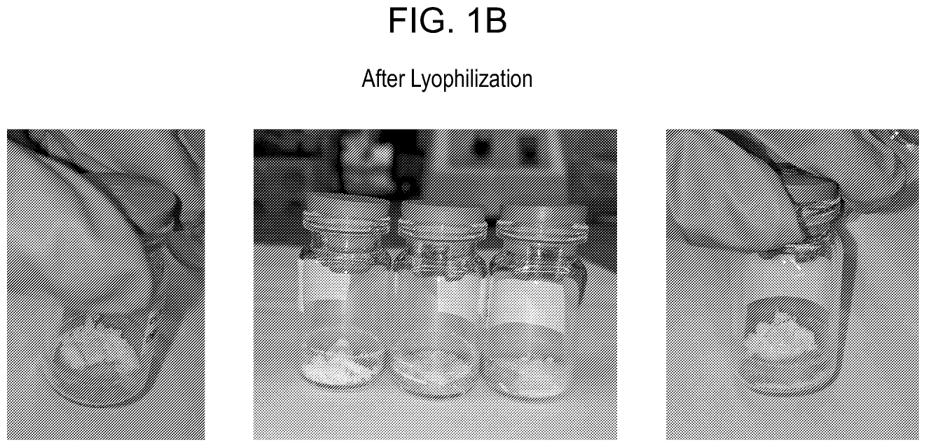

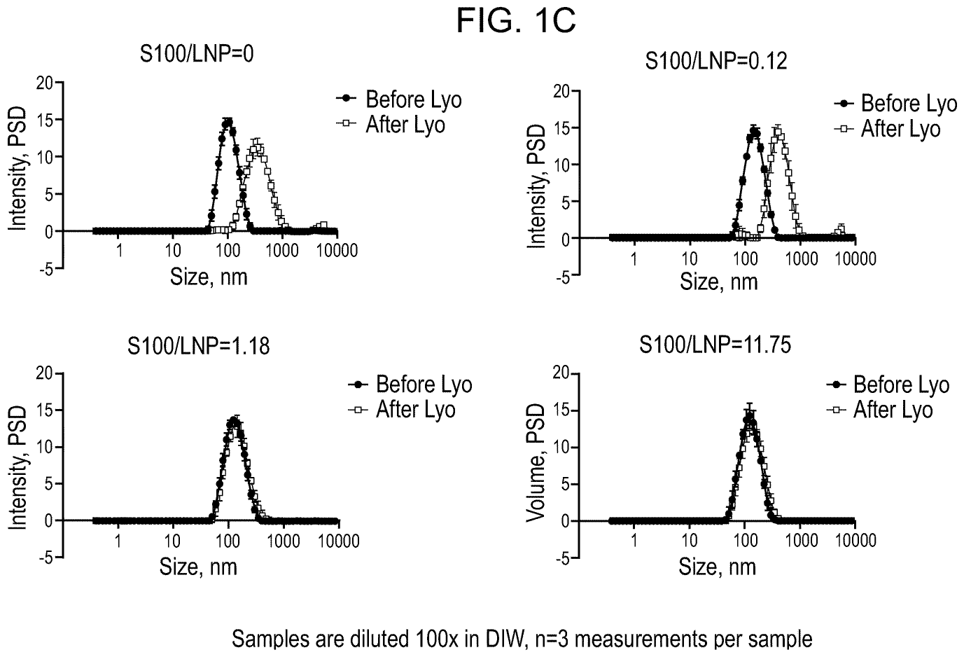

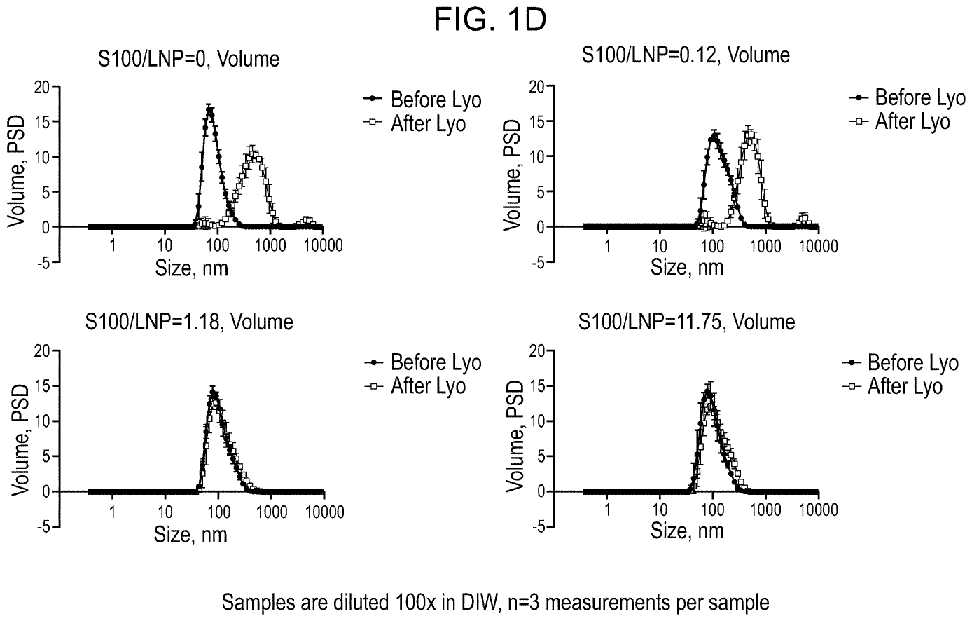

For a trial run, an old batch of LNP with 1×PBS was used. This LNP was buffer exchanged with 50 mM HEPES and used for the experiment. Samples were analyzed before lyophilization before the addition of 40% sucrose. After lyophilization, samples were reconstituted using the same volume of DIW as the LNP used originally (in this case 0.5 mL). The size and charge data are depicted in Table 6 and FIG. 1.

TABLE 6
S100-LNP analyzed for size (100x dil in DIW), and charge (10x dil
in DIW) before (without 40% sucrose), and after lyophilization

| S100/LNP | Before Lyophilization | | | After Lyophilization | | |
|---|---|---|---|---|---|---|
| (w/w) | Size, nm | PDI | ZP, mV | Size, nm | PDI | ZP, mV |
| 0 | 100.3 | 0.140 | 14.2 | 321.1 | 0.237 | 14.9 |
| 0.12 | 138.4 | 0.114 | 5.7 | 381.6 | 0.259 | −1.4 |
| 1.18 | 118.5 | 0.128 | −12.5 | 129.4 | 0.148 | −26.3 |
| 11.75 | 110.9 | 0.144 | −31.5 | 122.4 | 0.167 | −33.5 |

### Systematic Addition of S100 to LNP

The following samples were prepared covering LNPs lyophilized with and without Sucrose and S100 as depicted in Table 7. The effective Sucrose concentration is 20% in the pre-lyophilized solution. In case of A, and E4, 200 μL and 209.8 μL of 50 mM HEPES was added in place of the 40% Sucrose solution.

TABLE 7
Systematic addition of S100 to the LNP

| | P/LNP | | | 40% Sucrose |
|---|---|---|---|---|
| Formulation | (w/w) | LNP, mL | S100, uL | Solution, uL |
| A | 0 | 0.20 | 0 | 0 |
| B | 0 | 0.20 | 0 | 200.0 |
| C | 0.12 | 0.20 | 0.9984 | 201.0 |
| D | 0.33 | 0.20 | 2.7456 | 202.7 |
| E1 | 1.18 | 0.20 | 9.8176 | 209.8 |
| E4 | 1.18 | 0.20 | 9.8176 | 0 |
| F | 3.33 | 0.20 | 27.7056 | 227.7 |
| G | 11.75 | 0.20 | 97.76 | 297.8 |

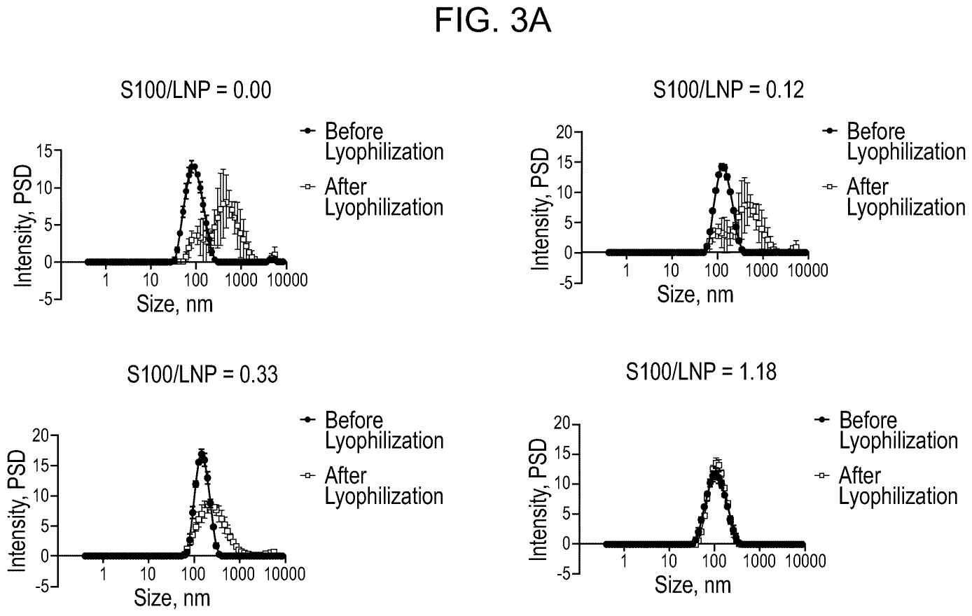

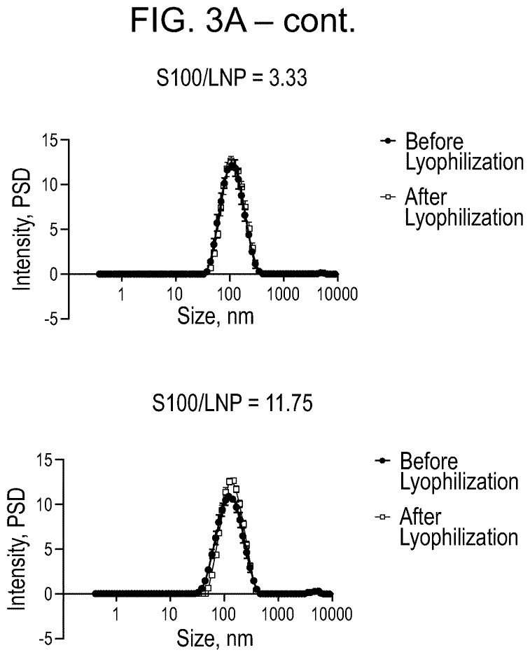

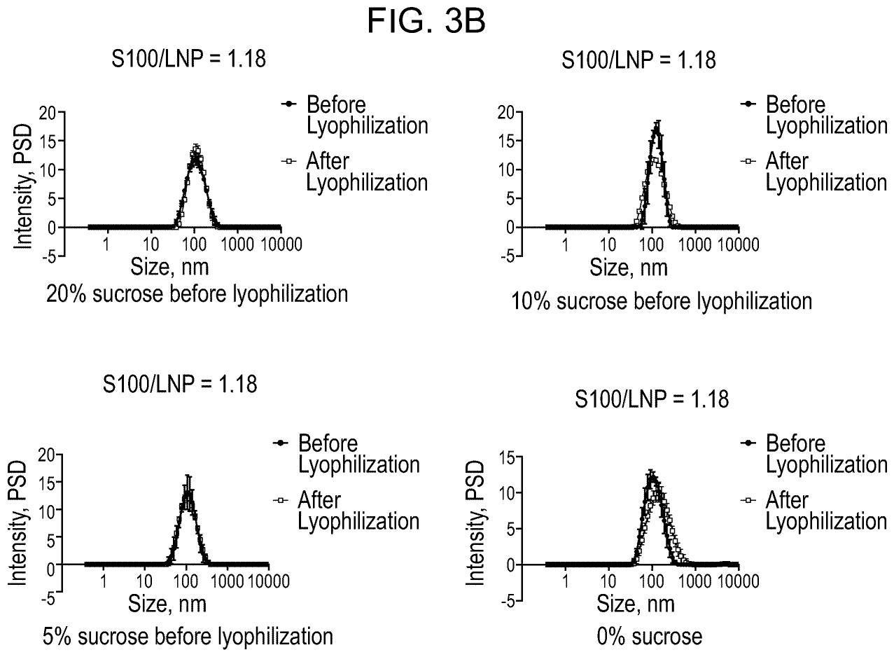

The size data is depicted in Table 8. As indicated, size doesn't change upon the addition of Sucrose. Upon lyophilization, the formulations were reconstituted with 200 μL of DIW. Formulation A which did not have any polymer or cryoprotectant, completely crashed out, with visible particles. Upon the addition of the polymer (P), formulations needed at least P/LNP=1.18 to suppress aggregation after reconstitution, as seen earlier (Table 6). Interestingly, it is also observed that E4 which had only S100 and no cryoprotectant (sucrose) was also able to inhibit size increase after lyophilization. The size changes can be further visualized in FIG. 3A.

TABLE 8
Size distribution and Charge of LNPs before and after lyophilization

| | | | | | | | | After Lyophilization: | | |
|---|---|---|---|---|---|---|---|---|---|---|
| | | LNP + S100 | | | LNP + S100 + Sucrose | | | LNP + S100 + Sucrose | | |
| | P/LNP | Size, | | ZP, | Size, | | ZP, | Size, | | ZP, |
| Formulation | (w/w) | nm | PDI | mV | nm | PDI | mV | nm | PDI | mV |
| A | 0 | 83.39 ± | 0.167 ± | 7.82 ± | — | — | — | Polydisperse | | 7.34 ± |
| | | 0.951 | 0.008 | 1.04 | | | | | | 0.588 |
| B | 0 | 83.39 ± | 0.167 ± | 7.82 ± | — | — | — | 284.9 ± | 0.470 ± | 7.78 ± |
| | | 0.951 | 0.008 | 1.04 | | | | 9.047 | 0.029 | 0.150 |
| C | 0.12 | 122.3 ± | 0.092 ± | 8.23 ± | 126.7 ± | 0.131 ± | 8.04 ± | 284.2 ± | 0.487 ± | 4.81 ± |
| | | 1.102 | 0.027 | 0.545 | 0.7371 | 0.020 | 0.877 | 10.45 | 0.002 | 0.418 |
| D | 0.33 | 136.2 ± | 0.085 ± | 7.03 ± | 141.0 ± | 0.104 ± | −1.47 ± | 213.9 ± | 0.303 ± | 2.44 ± |
| | | 0.3606 | 0.006 | 1.19 | 2.230 | 0.034 | 0.295 | 5.992 | 0.034 | 0.652 |
| E1 | 1.18 | 92.44 ± | 0.148 ± | −38.3 ± | 97.13 ± | 0.166 ± | −37.8 ± | 103.6 ± | 0.135 ± | −36.1 ± |
| | | 1.023 | 0.020 | 1.39 | 0.9455 | 0.026 | 0.666 | 1.955 | 0.011 | 0.839 |
| E4 | 1.18 | 92.44 ± | 0.148 ± | −38.3 ± | — | — | — | 127.9 ± | 0.225 ± | −35.3 ± |
| | | 1.023 | 0.020 | 1.39 | | | | 0.5196 | 0.018 | 0.757 |
| F | 3.33 | 99.27 ± | 0.170 ± | −44.8 ± | 101.3 ± | 0.170 ± | −40.0 ± | 107.2 ± | 0.165 ± | −47.2 ± |
| | | 0.7181 | 0.011 | 1.33 | 1.73 | 0.019 | 0.981 | 1.415 | 0.009 | 1.27 |
| G | 11.75 | 112.2 ± | 0.193 ± | −48.1 ± | 110.17 ± | 0.227 ± | −51.0 ± | 119.2 ± | 0.209 ± | −45.9 ± |
| | | 2.352 | 0.010 | 0.6 | 1.07 | 0.013 | 0.709 | 0.8021 | 0.012 | 1.64 |

### Effect of Sucrose on Lyophilization

While lyophilization is a great solution to improve the stability of the LNPs, it can have multiple uses particularly for oral delivery by enabling capsule filling, higher concentrations, matrixing in gels to enable sustained release, to name a few. To concentrate, the amount of diluent to be added would be a lot lower than the pre-lyophilization volume, making it difficult to dissolve the higher solid content, and the resulting higher viscosity that would affect dosing. The physico-chemical effects of the amount of sucrose were assessed by adding different concentrations of sucrose to LNP+S100 at 1.18 (w/w) P/LNP as depicted in Table 7 and visualized in FIG. 3B. Interestingly, the amount of sucrose in the formulation doesn't have a statistically significant effect on the size characteristics after lyophilization. In fact, as in the case of E4, the sample seems to be decently protected despite having no sucrose.

TABLE 9
Size Distribution and charge as a function of Sucrose addition
before and after lyophilization of 50 mM HEPES dialyzed LNP

| | Effective | | | | | | |
|---|---|---|---|---|---|---|---|
| | Sucrose | | | | After Lyophilization: | | |
| | concentration in | LNP + S100 + Sucrose | | | LNP + S100 + Sucrose | | |
| | pre-lyophililzed | Size, | | ZP, | Size, | | ZP, |
| Formulation | solution | nm | PDI | mV | nm | PDI | mV |
| E1 | 20% | 97.13 ± | 0.166 ± | −37.8 ± | 103.6 ± | 0.135 ± | −36.1 ± |
| | | 0.9455 | 0.026 | 0.666 | 1.955 | 0.011 | 0.839 |
| E2 | 10% | 117.8 ± | 0.077 ± | −37.1 ± | 106.1 ± | 0.162 ± | −36.0 ± |
| | | 12.41 | 0.019 | 0.794 | 1.916 | 0.004 | 0.954 |
| E3 | 5% | 100.82 ± | 0.149 ± | −36.6 ± | 102.4 ± | 0.153 ± | −35.5 ± |
| | | 4.105 | 0.052 | 1.06 | 0.5686 | 0.007 | 0.569 |
| E4 | 0 | 92.44 ± | 0.148 ± | −38.3 ± | 127.9 ± | 0.225 ± | −35.3 ± |
| | | 1.023 | 0.020 | 1.39 | 0.5196 | 0.018 | 0.757 |

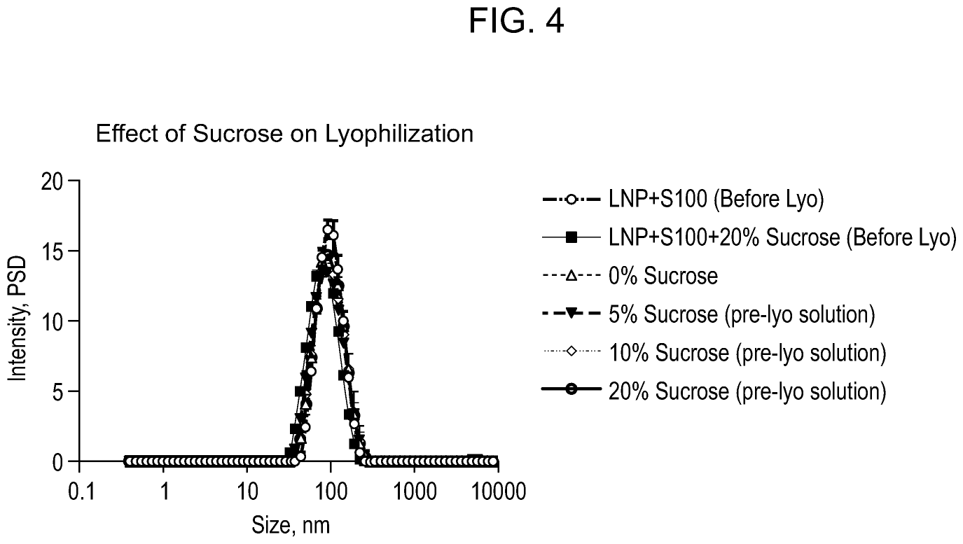

The same was observed for LNP dialyzed against 1×PBS as depicted in Table 10 and FIG. 4.

TABLE 10
Size Distribution and charge as a function of Sucrose addition
before and after lyophilization of 1x PBS dialyzed LNP

| | Effective | | | | | | |
|---|---|---|---|---|---|---|---|
| | Sucrose | | | | | | |
| Formulation: | concentration | | | | After Lyophilization: | | |
| S100/1x PBS | in pre- | LNP + S100 + Sucrose | | | LNP + S100 + Sucrose | | |
| LNP = 3.33 | lyophilized | Size, | | ZP, | Size, | | ZP, |
| (w/w) | solution | nm | PDI | mV | nm | PDI | mV |
| E1 | 20% | 82.57 | 0.148 | −38.8 | 90.15 | 0.115 | −35.5 |
| E2 | 10% | | | | 87.40 | 0.13 | −33.70 |
| E3 | 5% | | | | 86.3 | 0.146 | −30.6 |
| E4 | 0 | 91.72 | 0.197 | −37.3 | 114.1 | 0.131 | −30.8 |

### In Vitro Cell Transfection

To test whether the lyophilized LNPs were still functional, RAW264.7 cells were transfected with the formulations to assess their effect on functionality. RAW264.7 cells were used because it is a hard to transfect cell line.

Measuring free siRNA using Ribogreen assay is challenging on formulations that contain S100 (negative charge interacts with the dye fluorescing it), so it was decided to go with the siRNA corresponding to the LNP content at the beginning of the lyophilization process. The LNP dialyzed against 50 mM HEPES contains 5698 nM of total siGAPDH, with 97.5% encapsulation.

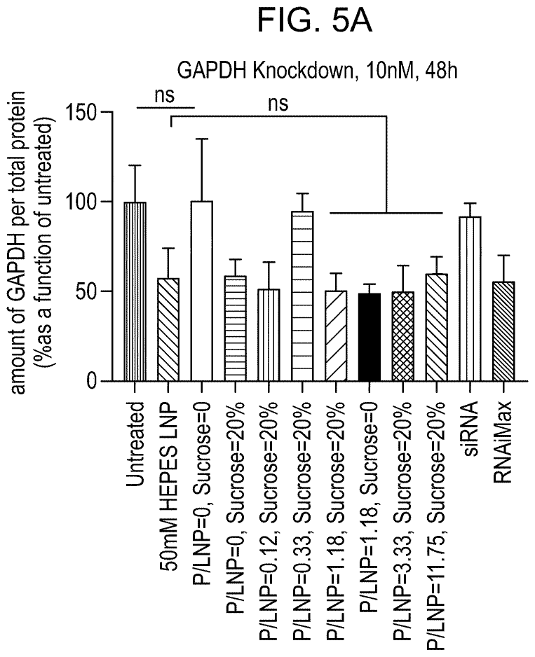

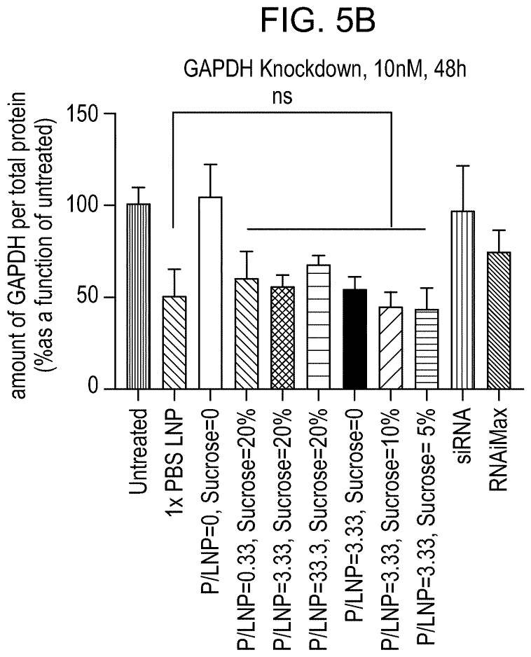

As can be seen from FIG. 5A, when compared to LNP, Formulation E was as functional after lyophilization as it was before lyophilization. Formulation A, that didn't have any S100 polymer, that crashed upon reconstitution, didn't show any functionality, just as the untreated formulation. Another study was conducted on LNPs dialyzed against 1×PBS and lyophilized with S100, tested on RAW264.7 cells (FIG. 5B). A similar trend was observed, where LNPs lyophilized with S100 were functional in comparison to LNPs lyophilized without the polymer as seen is FIG. 5B.

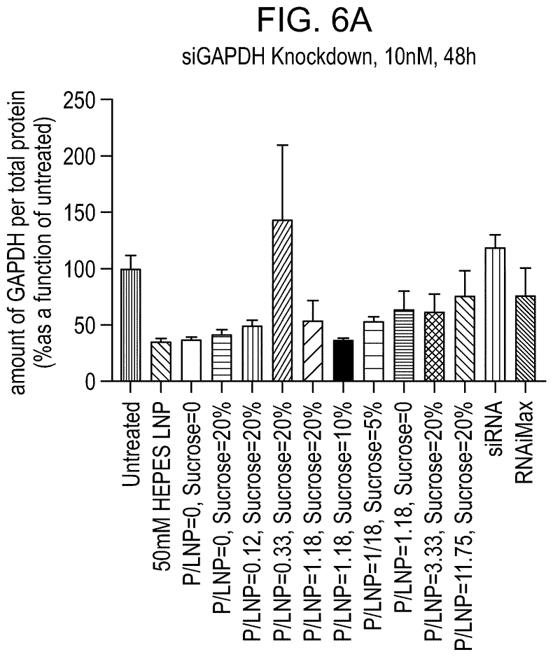

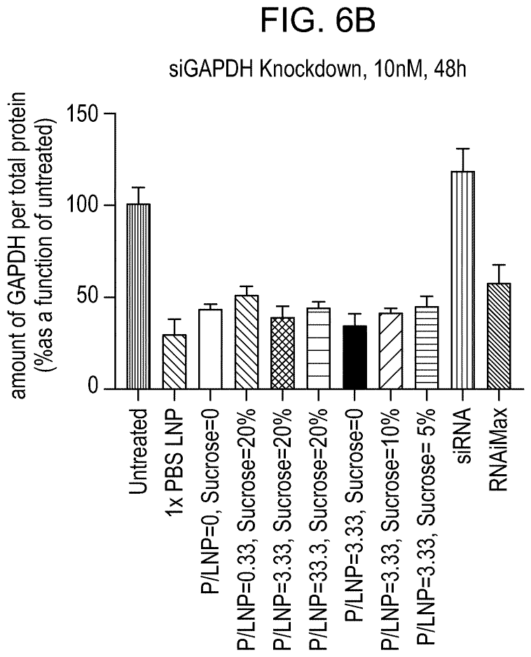

A similar trend was observed in HeLa cells despite most formulations being reconstituted 1 day earlier as is depicted in FIGS. 6A and 6B.

As is apparent from FIGS. 6A and 6B, weight ratios of ionic polymer to LNPs of 1.18, 3.33, 11.75 and 33.3 were effective in substantially silencing the GAPDH gene, showing substantial reductions in GAPDH production from untreated cells. When dialyzed against 50 mM HEPES, samples with a weight ratio of ionic polymer to LNPs of 1.18 with sucrose concentrations of 0%, 10%, 20% and 40% were effective in reducing GAPDH production by about 40% or more. When dialyzed against 1×PBS, samples with a weight ratio of ionic polymer to LNPs of 3.33 with sucrose concentrations of 0%, 10%, 20% and 40% were effective in reducing GAPDH production.

### Example 2

### Physicochemical Properties and Efficacy

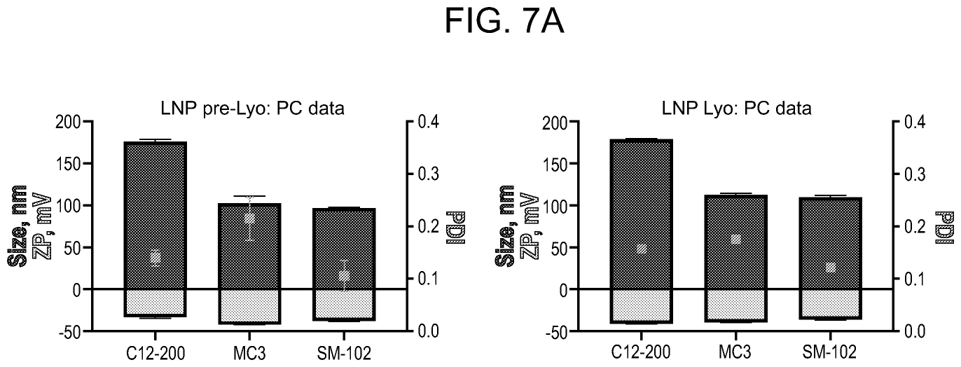

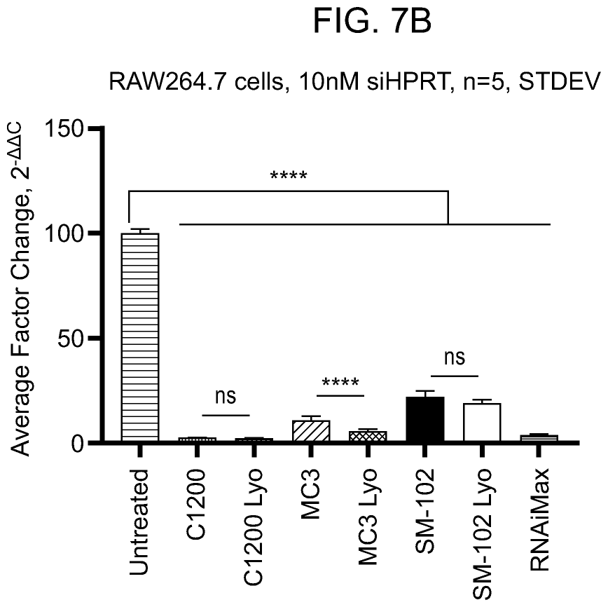

siHPRT LNPs were prepared using MC3 and SM-102 and tested in vitro in RAW 264.7 cells for knock down comparing LNPs vs PCLNPs of these variants. The data showed that the formulations retained their potency after lyophilization, indicating the universality of the technology. The physico-chemical data after adding the polymer, before and after lyophilization, and efficacy comparison between LNP and PCLNP is depicted in FIGS. 7A and 7B, respectively.

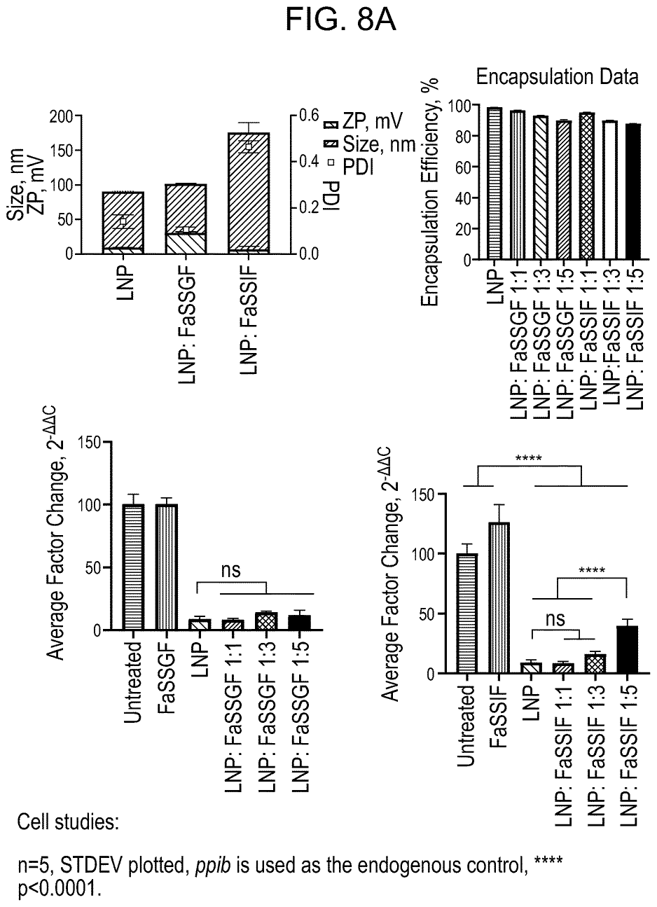

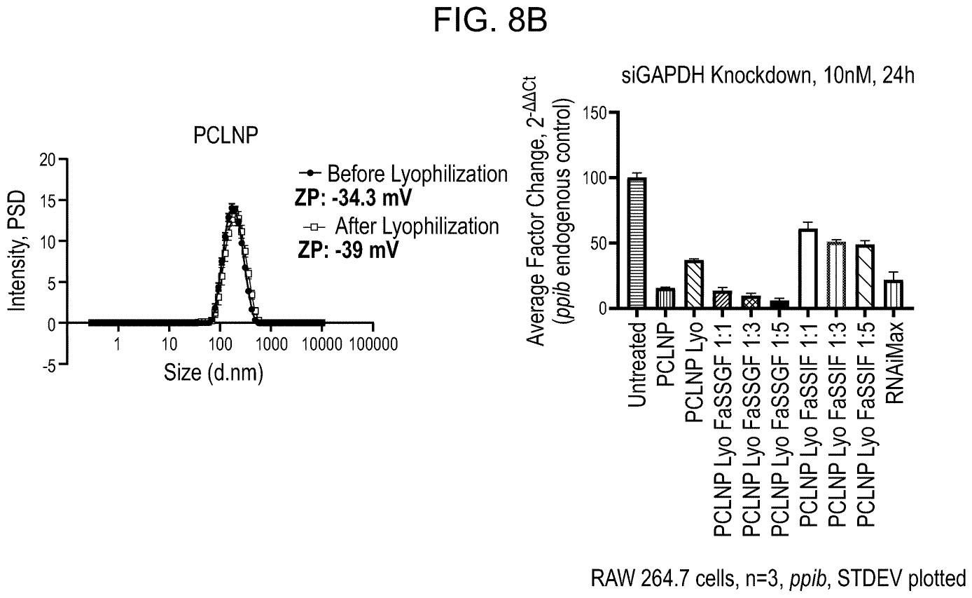

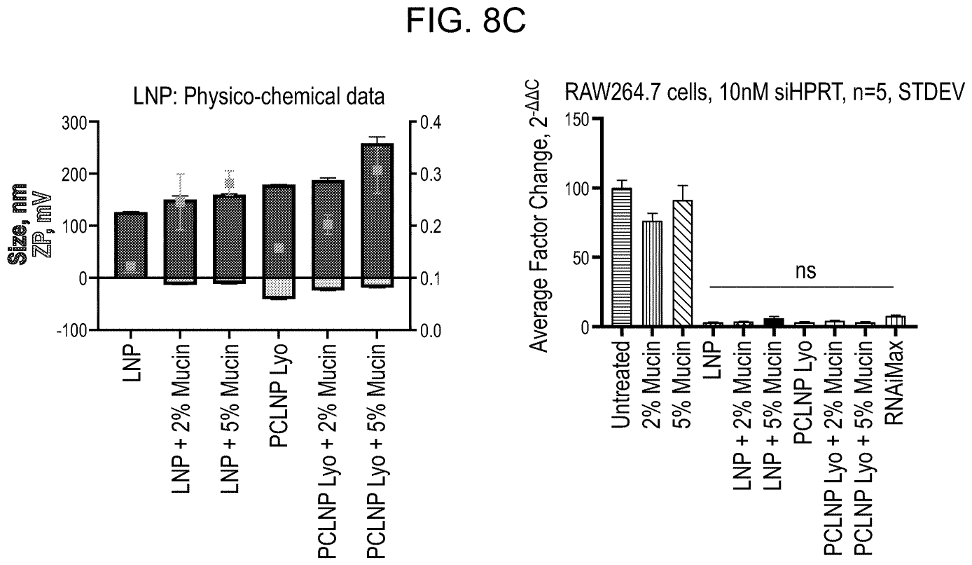

Additionally, to test out the effectiveness of the LNPs and PCLNPs after subjecting them to biorelevant media pertaining to the GI tract, such as Fasted Gastric and Intestinal Fluid, and Mucin, we observed retention of efficacy as depicted in FIGS. 8A, B, and C. In FIG. 8A, LNP stability under mouse GI conditions of volume dilutions of 1:1 to 1:5 dilution in Fasted Stimulated Gastric Fluid (FaSSGF) and in Fasted Stimulated Intestinal Fluid (FaSSIF) for 1 h at 37° C. (a) Representative Physicochemical data shows bimodal distribution in FaSSIF (1:5 dilution) due to the bile micelles and higher positive charge in Gastric media (1:3 dilution) due to ionization of positive charge. (b) Encapsulation Data shows good stability after 1 hour of incubation at 37° C. (d) GAPDH mRNA Expression in RAW264.7 cells after incubation in (c) FaSSGF and (d) FaSSIF for 1 h at 37° C. FIG. 8B depicts the physicochemical properties and in vitro efficacy data when the PCLNPs after lyophilization are incubated with FaSSGF and FaSSIF and the transfection is assessed on cells. In FIG. 8C, we observe the retention of potency of the LNPs and PCLNPs after lyophilization, after being incubated with mucin.

### Example 3

In Vivo Oral Delivery (siRNA)

LNPs were tested for in vivo efficacy to knock down endogenous HPRT. The LNP formulation encompassing siHPRT was prepared as Example 1, except that the S100 addition and Lyophilization were not performed. C12-200 and DOTMA were used as ionizable lipid and cationic lipid respectively.

2 mg/kg of siHPRT encapsulated LNP was administered in SKH1 female mice after overnight fasting. Food was introduced 6 hours after dosing. Animals were necropsied at 24 h and 48h. All the GI organs were extracted, and mRNA levels were quantified by RT-qPCR using GAPDH as the endogenous gene, for 24 h and 48 h timepoints.

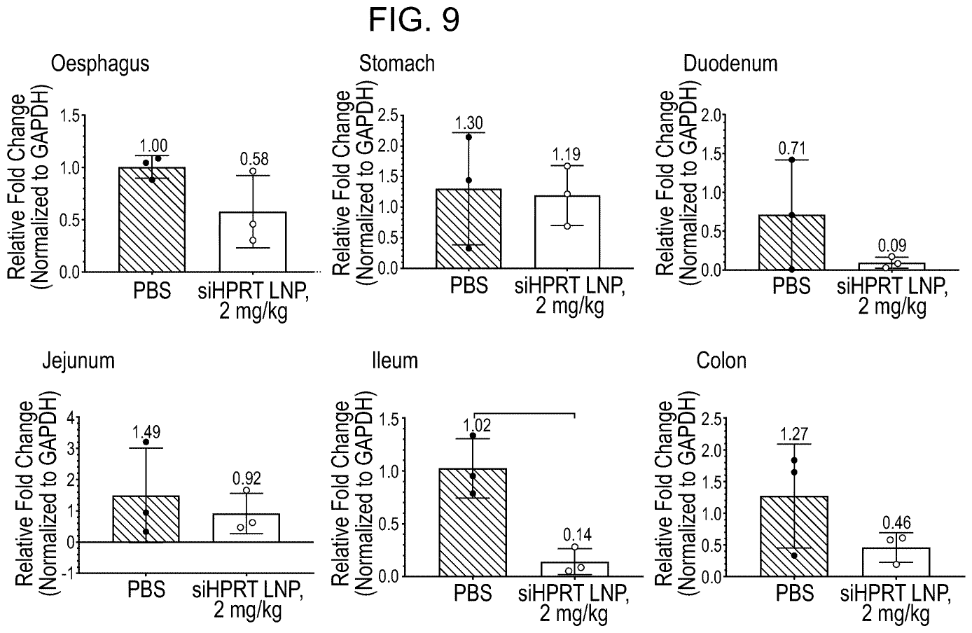

While HPRT knock down wasn't observed at 24 h, 48 h samples trend in favor of gene knockdown with statistically significant knockdown observed in the ileum (FIG. 9).

### Example 4

In Vivo Oral Delivery (mRNA)

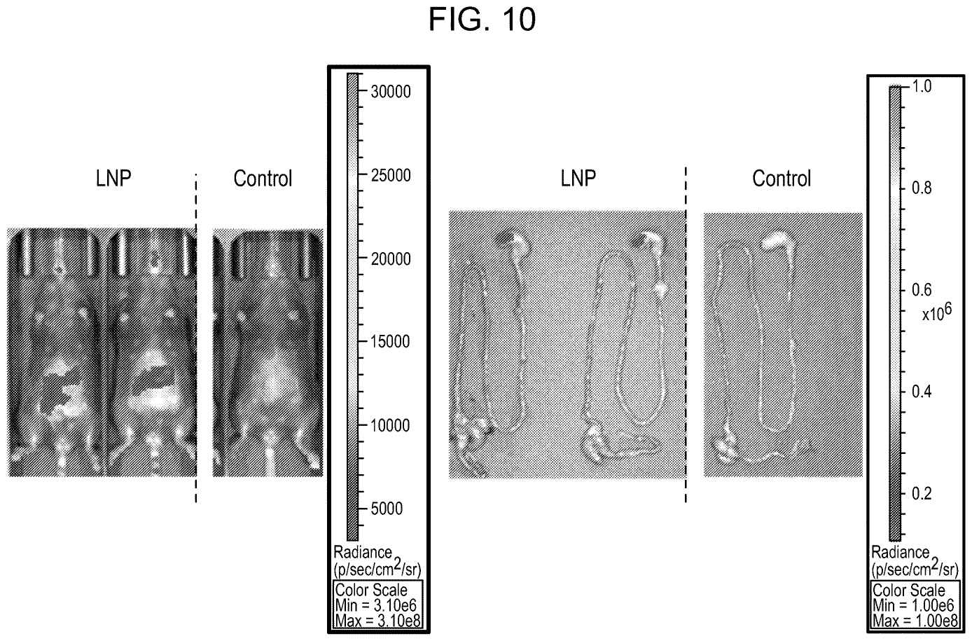

LNPs were tested for in vivo efficacy of expression of mRNA. The LNP formulation encompassing FLuc mRNA was prepared as Example 3 except that the FLuc mRNA is used instead of siHPRT. Food was introduced 1 hour after dosing instead of 6h. A dose corresponding to 2 mg/kg of FLuc mRNA encapsulated in the LNP was administered orally. PBS was administered orally to the control mice. IVIS data showed luminesce in the gastrointestinal tract the LNPs in comparison to the control animals. Upon organ isolation and subsequent IVIS, luminescence was only seen in the LNPs arm (FIG. 10).

### Example 5

In Vivo Oral Delivery (siRNA)

LNPs were tested for in vivo in an acute DSS-colitis mouse model. The LNP formulation encompassing siRNA against TNF-α (siTNF) was prepared as Example 3 except that the siTNF is used instead of siHPRT. The siTNF(CG) (Chemegenes, Sense strand: GCUCUUCUGUCUACUGAACUU, Anti-sense strand: AAGUUCAGUAGACAGAAGAGCGU) was used as the siTNF.

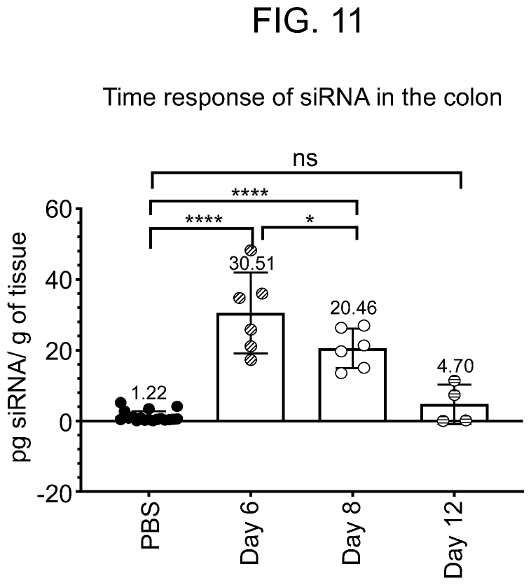

B6/J mice were administered with 2% DSS in their drinking water for 12 days. The first 5 days of DSS treatment, treatment groups were administered with the LNP. The total dose of 10 mg/kg of siRNA was administered over the 5 days of treatment. The untreated control was administered with PBS instead of LNPs. On Day 6,8, and 12, the DSS only group and DSS+siTNF(CG) LNP group were assessed to detect the siRNA in the colon. Stemloop qPCR was used to quantify the siRNA within the tissues. FIG. 11 shows the presence of siRNA in the colon on Day 6 and Day 8, and baseline levels observed on Day 12. Day 6 and Day 8 siRNA contents indicate the reduction in siRNA content with time.

We claim:

1. A composition comprising at least one lipid nanoparticle encapsulating a therapeutic agent and an aqueous solution comprising a salt and an anionic polymer, wherein the salt and the anionic polymer are dissolved in the aqueous solution.

2. The composition of claim 1, wherein the anionic polymer is bound to the surface of the lipid nanoparticle, thereby forming a polymer coated lipid nanoparticle (“PCLNP”).

3. The composition of claim 2, wherein the polymer coat covers at least 10%, at least 20%, at least 30%, at least 40%, at least 50%, at least 60%, at least 70%, at least 80%, at least 90%, at least 95%, at least 98%, or at least 100% of the surface of the lipid nanoparticle.

4. The composition of any one of claims 1 to 3, wherein the anionic polymer is a methacrylic acid polymer.

5. The composition of claim 2, wherein the PCLNP has a negative zeta potential.

6. The composition of claim 5, wherein the zeta potential is between about −0.1 mV to about −50 mV, about −1.0 mV to about −45 mV, about −2.0 mV to about −30 mV, about −10 mV to about −20 mV, or about −5.0 mV to about −10 mV.

7. The composition of claim 5, wherein the zeta potential is at least −0.1 mV, at least −0.5 mV, at least −1.0 mV, at least −2.0 mV, at least −5.0 mV, at least −10 mV, at least −15 mV, at least −20 mV, at least −25 mV, at least −30 mV, at least −35 mV, at least −40 mV, at least −45 mV, or at least −50 mV.

8. The composition of claim 5, wherein the zeta potential is at most −0.1 mV, at most −0.5 mV, at most −1.0 mV, at most −2.0 mV, at most −5.0 mV, at most −10 mV, at most −15 mV, at most −20 mV, at most −25 mV, at most −30 mV, at most −35 mV, at most −40 mV, at most −45 mV, or at most −50 mV.

9. The composition of any one of claims 5-8, wherein the zeta potential is between about −10 mV to about −50 mV.

10. The composition of any one of claims 1 to 9, wherein the polydispersity index (PDI) of the PCLNPs in the composition is less than 1.0, less than 0.9, less than 0.8, less than 0.7, less than 0.6, less than 0.5, less than 0.4, less than 0.3, less than 0.2, or less than 0.1.

11. The composition of any one of claims 1 to 10, wherein the PDI is less than 0.2.

12. The composition of any one of claims 1 to 11, wherein the difference of the size of the PCLNP before and after the lyophilization is less than 40%, less than 35%, less than 30%, less than 25%, less than 20%, less than 15%, less than 10%, or less than 5%.

13. The composition of any one of claims 1-12, wherein the difference of the PDI of the PCLNP before and after the lyophilization is less than 40%, less than 35%, less than 30%, less than 25%, less than 20%, less than 15%, less than 10%, or less than 5%.

14. The composition of any one of claims 1-13, wherein the nucleic acid molecule is DNA or RNA.

15. The composition of claim 14, wherein the nucleic acid molecule is RNA.

16. The composition of claim 14 or 15, wherein the RNA is mRNA, microRNA (miRNA), or short interfering RNA (siRNA).

17. The composition of any one of claims 1-16, wherein the nucleic acid molecule is between about 10 nucleotides (nt) and 20 kilobases (kb).

18. The composition of any one of claims 1-17, wherein the lipid nanoparticle comprises at least one lipid selected from the group consisting of a cationic lipid, an anionic lipid, a neutral lipid, a zwitterionic lipid, an ionizable lipid, a helper lipid, a cholesterol, a PEG-lipid, phospholipid, and a combination thereof.

19. The composition of claim 18, wherein the lipid nanoparticle comprises an ionizable lipid and a cationic lipid.

20. The composition of claim 18 or 19, wherein the combination of the ionizable lipid and the cationic lipid is more than about 20%, more than about 30%, more than about 40%, more than about 50%, or more than about 60% of the total lipid in the lipid nanoparticle.

21. The composition of any one of claims 18-20, wherein the ionizable lipid is a primary amino lipid, a secondary amino lipid, a tertiary amino lipid, or a multivalent lipid.

22. The composition of any one of claims 18-21, wherein the cationic lipid is a quaternary amino lipid.

23. The composition of any one of claims 18-21, wherein the cationic lipid is 1,2-di-O-octadecenyl-3-trimethylammonium propane (DOTMA) or 1,2-dioleoyl-3-trimethylammonium-propane (DOTAP).

24. The composition of any one of claims 1-23, wherein the lipid nanoparticle net charge is negative at pH 7.

25. The composition of any one of claims 1-23, wherein the lipid nanoparticle net charge is positive at pH 7.

26. The composition of any one of claims 1-23, wherein the lipid nanoparticle net charge is neutral at pH 7.

27. The composition from any one of the previous claims, wherein the aqueous solution comprising a salt is selected from the group consisting of HEPES (4-(2-hydroxyethyl)-1-piperazineethanesulfonic acid), PBS (phosphate buffered saline), TRIS (tris(hydroxymethyl)aminomethane), MES (2-(N-morpholino)ethanesulfonic acid), and citrate buffer.

28. The composition of claim 27, wherein the aqueous solution comprising a salt is HEPES.

29. The composition of claim 27, wherein the aqueous solution comprising a salt is PBS.

30. The composition of any one of claims 27 to 29, wherein the aqueous solution comprising a salt has a pH of about pH 7.0 to about pH 8.0.

31. The composition of claim 30, wherein the aqueous solution comprising a salt has a pH of about 7.6.

32. The composition of any one of the previous claims, wherein the composition further comprises a sugar.

33. The composition of claim 32, wherein the sugar is disaccharide.

34. The composition of claim 32 or 33, wherein the sugar is selected from the group consisting of lactose, sucrose, trehalose, and a combination thereof.

35. The composition of any one of claims 32 to 34, wherein amount of sugar is between about 0% and about 40%.

36. The composition of any one of claims 32 to 35, wherein the amount of sugar is at least 5%, least 10%, at least 20%, or at least 30%.

37. The composition of any one of claims 32 to 36, wherein the amount of sugar is about 5%.

38. The composition of any one of the previous claims, wherein the nucleic acid molecule is a short interfering RNA, the anionic polymer is a methacrylic acid polymer, and the lipid nanoparticle comprises at least one lipid selected from the group consisting of a cationic lipid, a polar lipid, a zwitterionic lipid, or a combination thereof.

39. The composition of any one of the previous claims, wherein the wherein the nucleic acid molecule is a short interfering RNA, the anionic polymer is a methacrylic acid polymer, and the lipid nanoparticle comprises at least one cationic lipid.

40. A pharmaceutical composition, comprising the composition of any one of claims 1-39, and a pharmaceutically acceptable carrier.

41. The composition of any one of claims 1-39 or the pharmaceutical composition of claim 38, wherein the composition is lyophilized.

42. A method of preserving the lyophilized composition of claim 41, comprising storing the lyophilized product at a temperature of about 2° C. to about 8° C.

43. A method of preserving the lyophilized composition of claim 41 comprising storing the lyophilized product at a temperature of about −20° C.

44. A method of reconstituting the lyophilized composition of claim 41 comprising adding a liquid medium to the lyophilized composition.

45. The method of claim 44, wherein the liquid medium is an aqueous medium.

46. A method of producing a pharmaceutical composition, comprising
a) producing lipid nanoparticles from at least one lipid and at least one nucleic acid molecule; and
b) mixing an aqueous solution comprising a salt and an anionic polymer with the lipid nanoparticles produced in step a) and a pharmaceutically acceptable carrier.

47. The method of claim 46, further comprising c) lyophilizing the resulting mixture produced in the step b).

48. The method of claim 46 or 47, wherein the anionic polymer is a methacrylic acid polymer.

49. A method of stabilizing a lipid nanoparticle for lyophilization, the method comprising:
a) producing lipid nanoparticles from the at least one lipid and the at least one nucleic acid molecule;
b) mixing an aqueous solution comprising a salt and an anionic polymer with the lipid nanoparticles produced in step a); and
c) lyophilizing the resulting mixture,
thereby stabilizing the lyophilized lipid nanoparticle.

50. The method of any one of claims 45 to 49, wherein the anionic polymer is dissolved in a buffer.

51. A method of manufacturing a stable pharmaceutical composition, the method comprising:
a) dissolving an anionic polymer in an aqueous solution comprising a salt; and
b) mixing the dissolved anionic polymer with a lipid nanoparticle comprising at least one nucleic acid.

52. The method of claim 51, further comprising c) lyophilizing the resulting mixture produced in the step b).

53. The method of any one of claims 46-52, wherein step b) further comprises a sugar.

54. The method of claim of 53, wherein the amount of sugar is about 5%.

55. The method of claim 51 or 52, wherein the nucleic acid molecule is DNA or RNA.

56. The method of claim 55, wherein the RNA is mRNA, microRNA (miRNA), or short interfering RNA (siRNA).

57. The method of any one of claims 46 to 56, wherein the lipid nanoparticle comprises an ionizable lipid and/or a cationic lipid.

58. The method of claim 57, wherein the combination of the ionizable lipid and the cationic lipid is more than about 20%, more than about 30%, more than about 40%, more than about 50%, or more than about 60% of the total lipid in the lipid nanoparticle.

59. The method of claim 57 or 58, wherein the ionizable lipid is primary amino lipid, secondary amino lipid, tertiary amino lipid or a multivalent lipid.

60. The method of any one of claims 57 to 59, wherein the cationic lipid is a quaternary amino lipid.

61. The method of any one of claims 57 to 60, wherein the cationic lipid is 1,2-di-O-octadecenyl-3-trimethylammonium propane (DOTMA) or 1,2-dioleoyl-3-trimethylammonium-propane (DOTAP).

62. The method of any one of claims 46 to 61, wherein the an aqueous solution comprising a salt is a buffer selected from the group consisting of HEPES (4-(2-hydroxyethyl)-1-piperazineethanesulfonic acid), PBS (phosphate buffered saline), TRIS (tris(hydroxymethyl)aminomethane), MES (2-(N-morpholino)ethanesulfonic acid), and citrate buffer.

63. The method of any one of claims 46 to 62, wherein the aqueous solution comprising a salt has a pH of about pH 7.0 to about pH 8.0.

64. A method of treating a disease or disorder in a subject, the method comprising administering the composition of any one of claims 1-39, the pharmaceutical composition of claim 40, or the lyophilized composition of claim 41 to the subject in need thereof.

65. The method of claim 64, wherein the disease or disorder is a disease or disorder of the gastrointestinal tract.

66. The method of claim 65, wherein the disease or disorder is Crohn's disease, irritable bowel syndrome, ulcerative colitis or gastrointestinal cancer.

67. The method of claim 64, wherein the lyophilized composition or the pharmaceutical composition is orally administered as a lyophilized admixture.

68. The method of claim 64, wherein the lyophilized composition or the pharmaceutical composition is intravenously administered as a lyophilized admixture.

69. A method of treating a disease or disorder in a subject comprising administering to the subject the composition of any one of claims 1-39, the pharmaceutical composition of claim 40, or the lyophilized composition of claim 41 reconstituted in a liquid medium.

70. The method of claim 69, wherein the reconstituted lyophilized composition is administered orally.

71. The method of claim 69, wherein the reconstituted lyophilized composition is administered intravenously.

72. The method of claim 69, wherein the reconstituted lyophilized composition is administered subcutaneously or intramuscularly.
